(19) United States
(12) Patent Application Publication
(10) Pub. No.: US 2023/0355811 A1
(43) Pub. Date: Nov. 9, 2023

(54) COMPOSITIONS INCLUDING MAGNETIC NANOPARTICLES AND METHODS OF USING AND MAKING THE SAME

(71) Applicant: University of Florida Research Foundation, Inc., Gainesville, FL (US)

(72) Inventors: Carlos Rinaldi, Gainesville, FL (US); Andreina Chiu Lam, Gainesville, FL (US)

(21) Appl. No.: 18/026,300

(22) PCT Filed: Sep. 28, 2021
(86) PCT No.: PCT/US2021/071614

§ 371 Date: Mar. 14, 2023

Related U.S. Application Data
(60) Provisional application No. 63/084,030, filed on Sep. 28, 2020.

(51) Int. Cl. A61K 49/18; A61B 5/055

(52) U.S. Cl. CPC A61K 49/186; A61B 5/055

(57) ABSTRACT

The present disclosure provides for compositions including coated magnetic particles (e.g., coated magnetic nanoparticles), methods of using the coated magnetic particles such as imaging a subject (e.g., a mammal), tissue, organ, or the like, a cryopreservation composition including the coated magnetic particles, methods of use of the cryopreservation composition in biomaterials (e.g., tissue, organ, and the like), methods of making the composition and cryopreservation composition, and the like.

### CLAIM OF PRIORITY TO RELATED APPLICATION

This application claims priority to co-pending U.S. provisional application entitled “MAGNETIC NANOPARTICLES AND METHODS OF USING AND MAKING THE SAME” having Ser. No. 63/084,030, filed on Sep. 28, 2020, which is entirely incorporated herein by reference.

### BACKGROUND

Cryopreservation by vitrification is an attractive technology to store organs or tissues. However, fast and uniform rewarming larger organs from the vitrified state is challenging. Technologies that achieve uniform, fast, controlled warming of cryopreserved organs would make cryopreservation by vitrification feasible for biobanking of whole organs.

### SUMMARY

The present disclosure provides for compositions including coated magnetic particles (e.g., coated magnetic nanoparticles), methods of using the coated magnetic particles such as imaging a subject (e.g., a mammal), tissue, organ, or the like, a cryopreservation composition including the coated magnetic particles, methods of use of the cryopreservation composition in biomaterials (e.g., tissue, organ, and the like), methods of making the composition and cryopreservation composition, and the like.

In one aspect, the present disclosure provides compositions, comprising a coated magnetic nanoparticle having: a magnetic core; a first PEG-silanization coating covalently attached to the magnetic core, wherein the first poly(ethylene glycol) (PEG)-silanization coating comprises a mixture of PEG silane and aminosilane; and a second PEG coating covering at least a part of the first PEG-silanization coating, wherein the second PEG coating comprises PEG group having at least one amine reactive group, the second PEG coating is attached to the first PEG-silanization coating via an amino group on the aminosilane and the amine reactive group on the second PEG coating.

In one aspect, the present disclosure provides for methods comprising contacting a biomaterial with the composition described above and herein; freezing the biomaterial; and rewarming the biomaterial. In addition, the present disclosure includes the product of this method.

In one aspect, the present disclosure provides for methods for imaging in a subject of organs, tissues, or cells comprising: introducing the composition described above or herein into organs, tissues, or cells of a subject; and imaging the organs, tissues, or cells with an imaging technique.

In one aspect, the present disclosure provides methods for producing a coated magnetic nanoparticle comprising: subjecting a magnetic core to a first composition comprising a mixture of poly(ethylene glycol)-silane (PEG-silane) group and aminosilane to form a first nanoparticle coated with a first PEG-silanization coating comprising a first ligand derived from the PEG-silane group, wherein the first ligand is covalently attached to the magnetic core, wherein the aminosilane is covalently bonded to the magnetic core; and subjecting the first nanoparticle to a second composition comprising a PEG group to form a coated magnetic nanoparticle further coated with a second layer comprising PEG group, wherein the PEG group having at least one amine reactive group, wherein the second PEG coating is attached to the first PEG-silanization coating via an amino group on the aminosilane and the amine reactive group on the second PEG coating. The present disclosure includes a composition made from this method and those described herein.

### BRIEF DESCRIPTION OF THE DRAWINGS

The present disclosure may be better understood with reference to the following figures.

FIGS. 1.1A-1D show colloidally stable SPIONs including a PEGsilane/APS coating backfilled with additional PEG. (FIG. 1.1A) Intensity weighted distribution of backfilled PEG-coated SPIONs in PBS 1×over 28 days. (FIG. 1.1B) Intensity weighted distribution of backfilled PEG-coated SPIONs in VS55 (no vitrification). (FIG. 1.1C) Intensity weighted distribution of backfilled PEG-coated SPIONs in VS55 after vitrification and nanowarming. (FIG. 1.1D) Arithmetic mean and standard deviation of the DLS hydrodynamic diameter for backfilled SPIONs in PBS 1×and VS55 pre- and post-vitrification and nanowarming over 28 days.

FIGS. 1.2A-1.2D show mCPAs with exceptionally high and controllable heating rates. (FIG. 1.2A) Warming of CPA and mCPA solutions with SPION concentration of 10 mgFe/mL in a water bath at 37° C. and AMF at 42.5 kA/m, 278 kHz. The nanowarming rate for mCPA in AMF is 321° C./min. (FIG. 1.2B) Nanowarming of mCPA (10 mgFe/mL) at different applied alternating magnetic field conditions demonstrates control of the nanowarming rate. (FIG. 1.2C) Nanowarming rates at different SPION concentrations in VS55. Temperature profiles at different concentrations of SPIONs. (FIG. 1.2D) Relation between nanowarming rate and SPION concentration.

FIGS. 1.3A-1.3B show SPIONs and mCPAs with low primary cardiomyocyte cytotoxicity. (FIG. 1.3A) Relative viability of primary cardiomyocytes incubated with PEG backfilled SPIONs. Viability reported as the percentage of live cells relative to total cells determined by Hoechst-PI. (FIG. 1.3B) Relative viability of primary cardiomyocytes incubated on ice for 1 hour with VS55/media mixtures and after gradual addition of VS55 and replacement with mCPA. Relative viability reported as the percentage of live cells relative to total cells by Hoechst-PI.

FIGS. 1.4A-1.4D show evaluation of heart perfusion with mCPA using MPI. (FIG. 1.4A) Representative photo of a heart well perfused with mCPA at 1 mgFe/mL. (FIG. 1.4B) Representative photo of a heart perfused in with mCPA at 1 mgFe/mL and then out with Custodiol® HTK. (FIG. 1.4C) Co-registered image of hearts with their respective MPI signals, including (left to right) heart perfused in with mCPA and then out with Custodiol® HTK, heart perfused in with mCPA, and control heart perfused with Custodiol® HTK. (FIG. 1.4D) SPION iron mass in whole hearts determined from quantification of the MPI signal for n=3 hearts perfused in and n=4 hearts perfused in with mCPA and out with Custodiol® HTK.

FIG. 1.5 shows procedure for the ex vivo evaluation of magnetic cryoprotecting agent (mCPA) perfusion and removal after vitrification and nanowarming. Hearts were removed from male rats and perfused with Custodiol® HTK solution. Then the heart was perfused with mCPA containing 5 mgFe/mL. The perfused heart was submerged in mCPA and vitrified in a mechanical freezer at a cooling rate of 15° C./min, followed by storage in liquid nitrogen for 1 week. Nanowarming was performed in an alternating magnetic field (AMF, 42.5 kA/m and 278 kHz, achieving heating rates above the critical warming rate of 50° C./min for VS55. The SPIONs were perfused out of the heart using Custodiol® HTK, followed by Magnetic Particle Imaging (MPI) to quantify the SPIONs remaining in the hearts.

FIGS. 1.6A-1.6C show evaluation of mCPA perfusion, vitrification, cryostorage, and nanowarming of whole rat hearts. (FIG. 1.6A) Example of successful perfusion with mCPA, vitrification to 77 K, cryostorage at 77 K for 1 week, nanowarming with AMF (42.5 kA/m, 278 kHz), and subsequent removal of mCPA with Custodiol® HTK. Optical and MPI images after perfusion with mCPA, vitrification, cryostorage, and nanowarming (top) and after removal of mCPA (bottom). Iron mass in the heart was determined using MPI. (FIG. 1.6B) Examples of failed attempts. Left: Example of non-uniform perfusion of mCPA due to obstruction of the right ventricle. Middle: Example of heart damage (red oval) due to rewarming using external heating (not nanowarming). Right: Example of heart damage (blue circle) due to non-uniform nanowarming caused by the heart not being completely immersed in mCPA. The heart did not perfuse well as observed from the color difference at the bottom of the heart compared to the top of the heart. (FIG. 1.6C) Use of MPI to distinguish uniform (left) and non-uniform (right) perfusion with mCPA. The heart shown in the right of FIG. 1.6C is the same as the heart shown in the left of FIG. 1.6B, which had an obstruction in the right ventricle.

FIGS. 1.7A-1.7D show characterization of superparamagnetic iron oxide nanoparticles used to formulate magnetic cryopreservation agent (mCPA) solutions. (FIG. 1.7A) Representative TEM image of particles after PEG coating. (FIG. 1.7B) Core diameter distribution obtained from TEM images (N=509 particles analyzed). (FIG. 1.7C) Magnetization curve for mCPA (10 mg_{Fe}/mL in VS55) at 77 K. (FIG. 1.7D) Magnetization curve at 77 K in a field range representative of the amplitude of the AMF used in nanowarming experiments, demonstrating remanence of 7.9 Am²/kg and coercivity of 3.2 kA/m.

FIG. 1.8 shows colloidal stability of PEG coated SPIONs in VS55, showing an increase in size and distribution over 7 days.

FIG. 1.9 shows evaluation of SPION distribution in hearts using magnetic particle imaging. Image of each heart and the signal intensity from MPI scaled to the same range. Hearts that were not perfused shows no signal. Hearts perfused in with mCPA shows high signal from the particles, a bright yellow signal. Hearts in which mCPA was perfused out, signal is low, faint blue color.

FIG. 1.10 shows histological evaluation of gross tissue damage due to perfusion with mCPA. Representative H&E images of whole transversal cross-sections from the center and bottom of rat hearts from control, perfused in, and perfused in & out group. H&E shows similar cytoarchitecture texture of the myocardium across all groups.

FIG. 1.11 shows histological evaluation of rat hearts after perfusion with mCPA. Representative H&E images at a higher magnification of different areas of the transversal cross-section of the center and bottom of the control, perfused in, and perfused in and out rat hearts. The cytoarchitecture texture of the myocardium of the rat hearts that are perfused in and perfused in and out appear to be similar to the normal rat heart.

FIG. 1.12 shows histological assessment of SPION distribution in rat hearts after perfusion with mCPA. Representative Prussian blue images at a high magnification of different areas of the transversal cross-section of the center and bottom of the control, perfused in, and perfused in and out rat heart. No blue stain is observed as expected in the control heart since it was not perfused with particles. Blue stain is observed at various locations within the interstitial space of the perfused in heart and no blue stain is observed as expected in the perfused in and out heart since the particles were perfused out with Custodiol® HTK.

FIG. 2.1 illustrates a tracer evaluation via transmission electron microscopy: FIG. 2. 1A) RL-1C. FIG. 2.1B) Ferucarbotran, and FIG. 2.1C) Synomag®-D.

FIG. 2.2 illustrates graphs of the magnetic characterization of RL-1C. FIG. 2.2A illustrates a MH curve at 300 K PEG coated RL-1 particles in water. FIG. 2.2B illustrates MH curves at 295, 305, 315K in TEGMA. FIG. 2.2C illustrates ZFC/FC at 10 Oe in TEGMA. FIG. 2.2D illustrates physical, hydrodynamic and magnetic diameter distribution.

FIG. 2.3 illustrates MPI properties of commercially available tracers and RL SPIONs. FIG. 2.3A illustrates PSF obtained using relax module in MOMENTUM™ scanner shows signal intensity of SPIONs. FIG. 2.3B illustrates the serial dilution shows linear relationship of iron mass and MPI signal for all three tracers in 2D high-sensitivity scan modes. FIG. 2.3C illustrates 2D MPI maximum intensity projection for 1 μgFe in 1 μL of solution for all three tracers.

FIG. 2.4 illustrates representative MPI/CT images at short and long time points and MPI signal intensity in heart and liver ROIs as a function of time for each tracer. Each animal is shown with different markers. Data was fitted to a nonlinear least square fit single compartment model to estimate blood circulation half-life.

FIG. 2.5.1 illustrates custom 3D-printed sample bed for phantom and SPION sample MPI studies. Dimensions of this custom-designed sample bed were derived from Magnetic Insight's 45 mL sample tube design and modified to fit the custom sample holders that were designed and 3D-printed for this study.

FIG. 2.5.2 illustrates vertical capillary tube holder designed and 3D-printed for LoD studies. Custom sample holders were designed in Onshape and 3D printed using Formlabs Clear V4 resin with the Form 3 SLA 3D printer.

FIG. 2.5.3 illustrates vertical 0.2 mL microcentrifuge tube holder designed and 3D-printed for MPI relax scan measurements. The design accommodates 7 total tubes, with the entire lengths of 4 tubes able to be seen from a side view through exposed spaces. These 4 exposed spaces are 27.91 mm in vertical height with a center spacing of 22.4 mm.

FIG. 2.5.4 illustrates a rendering of two-part animal bed assembly designed and 3D-printed for MPI animal studies. The top and bottom animal bed parts (left) assemble a whole animal bed (right) that can be installed on Magnetic Insight's MPI scanner. The top part was designed with a flat bottom for ease of transfer of the mice from MPI to CT. The bottom part houses an integrated tubing system for isoflurane administration during imaging.

FIG. 2.5.5 illustrates magnetic characterization of ferucarbotran. FIG. 2.5.5A illustrates a MH curve at 300 K for ferucarbotran in water. FIG. 2.5.5B illustrates MH curves at 295, 305, 315K for ferucarbotran in TEGMA. FIG. 2.5.5C illustrates ZFC/FC at 10 Oe for ferucarbotran in TEGMA.

FIG. 2.5.6 illustrates the magnetic characterization of Synomag®-D. FIG. 2.5.6A illustrates a MH curve at 300 K for Synomag®-D in water. FIG. 2.5.6B illustrates MH curves at 295, 305, 315K for Synomag®-D in TEGMA. FIG. 2.5.6C illustrates ZFC/FC at 10 Oe for Synomag®-D in TEGMA.

FIG. 2.5.7 illustrates physical, magnetic, and hydrodynamic size distribution histograms for: FIG. 2.5.7A, ferucarbotran, FIG. 2.5.7B, Synomag®-D, FIG. 2.5.7C, RL-1A, FIG. 2.5.7D, RL-1B, and FIG. 2.5.7E, RL-1C.

FIG. 2.5.8 illustrates dynamic magnetic susceptibility characterization of relaxation mechanism for all three tracers. FIG. 2.5.8A illustrates ferucarbotran, FIG. 2.5.8B illustrates Synomag®-D, and FIG. 2.5.8C illustrates RL-1C in water (red circle) or water and glycerol mixture (blue triangle, 65 wt % of glycerol). Dash line indicating peak frequency position for each particle assuming Brownian Relaxation dominant, based on their hydrodynamic diameter.

FIG. 2.5.9 illustrates PSF for several RL-1 tracer batches demonstrate reproducibility in MPI performance.

FIG. 2.5.10 illustrates representative 2D MPI z-channel dilution series for commercially available tracers and RL-1C. MPI scans were acquired in high-sensitivity.

FIG. 2.5.11 illustrates mean signal to noise ratio (mSNR) as a function of tracer mass.

FIG. 2.5.12 illustrates collage showing representative MPI images to each time point for one mouse for RL-1C tracer. The co-registered optical image with MPI is for reference to the signals observed in the MPI scans at different times. The ROIs for the heart and liver/spleen were all the same size across all times

### DETAILED DESCRIPTION

### Definitions

For convenience, before further description of the present invention, certain terms used in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The articles “a,” “an” and “the” are used to refer to one or to more than one (i.e., to at least one) of the grammatical object of the article.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, the terms “optional” or “optionally” means that the subsequently described event or circumstance can or cannot occur, and that the description includes instances where said event or circumstance occurs and instances where it does not.

The term “alkyl” as used herein refers to a linear or branched saturated hydrocarbon. Examples of alkyl groups include, but are not limited to, methyl, ethyl, propyl such as propan-1-yl, propan-2-yl (isopropyl), butyls such as butan-1-yl, butan-2-yl (sec-butyl), 2-methyl-propan-1-yl (iso-butyl), 2-methyl-propan-2-yl (tert-butyl), pentyls, hexyls, octyls, and decyls. In some embodiments, an alkyl group has from 1 to 6 carbon atoms (C1-C6 alkyl).

As used interchangeably herein, “subject,” “individual,” or “patient” can refer to a vertebrate organism, such as a bird, reptile, amphibian, mammal (e.g. human, canine, feline, equine, cattle, etc.). In an aspect, the subject is a human. In an aspect, the subject is a domesticated animal such as a dog or cat. “Subject” can also refer to a cell, a population of cells, a tissue, an organ, or an organism, preferably to human and constituents thereof.

By “administration” is meant introducing an embodiment of the present disclosure into a subject. Administration can include routes, such as, but not limited to, intravenous, oral, topical, subcutaneous, intraperitoneal, intraarterial, inhalation, vaginal, rectal, nasal, introduction into the cerebrospinal fluid, or instillation into body compartments can be used. A preferred route is oral administration.

In accordance with the present disclosure, “a detectably effective amount” of the agent (e.g., coated magnetic nanoparticle) of the present disclosure is defined as an amount sufficient to yield an acceptable image after introduction of the agent. The detectably effective amount of the agent of the present disclosure can vary according to factors such as disease type, type of agent, and the like. Detectably effective amounts of the agent of the present disclosure can also vary according to instrument and digital processing related factors. Optimization of such factors is well within the level of skill in the art.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### DESCRIPTION

Aspects of the present disclosure provide for compositions including coated magnetic particles (e.g., coated magnetic nanoparticles), methods of using the coated magnetic particles such as imaging a subject (e.g., a mammal), tissue, organ, or the like, a cryopreservation composition including the coated magnetic particles, methods of use of the cryopreservation composition in biomaterials (e.g., tissue, organ, and the like), methods of making the composition and cryopreservation composition as well as the composition and cryopreservation composition resulting from the methods, and the like.

In an aspect, embodiments of the present disclosure are advantageous since the composition including the coated magnetic particles can have a long blood circulation half-life (e.g., about 7 hours) and are suitable for blood pool imaging applications and other applications where long blood circulation time is desirable during imaging. Additional details are provided in Example 2.

In another aspect, embodiments of the present disclosure are advantageous in that the composition including coated magnetic particles (also referred to as “magnetic cryoprotecting agent (mCPA)”) can have fast heating rates that are controllable through the magnitude of the applied alternating magnetic field and the specific type of composition used. Embodiments of the present disclosure can uniformly perfuse whole organs and be efficiently removed after vitrification and nanowarming. Additional details are provided in Example 1.

An embodiment of the composition can include a coated magnetic nanoparticle. A first poly(ethylene glycol) PEG-silanization coating is covalently attached (e.g., directly or indirectly) to a magnetic core. The first (PEG)-silanization coating can include a mixture of a PEG silane and aminosilane. In addition, the coating layers include a second PEG coating covering at least a part of the first PEG-silanization coating. The second PEG coating can include second PEG groups that each can have at least one amine reactive group and optionally a secondary PEG moiety. The second PEG coating is attached to the first PEG-silanization coating via an amino group on the aminosilane and the amine reactive group on the second PEG coating (e.g., the aminosilane bonded to the second PEG group). In an aspect, the molar ratio of PEG silane and aminosilane is about 2:1 to 1:2 or about 1:1. In an aspect, the molar ratio of PEG-silanization coating and second PEG coating is about 1:50 to 50:1, about 1:20 to 20:1, about 1:10 to 10:1, about 1:5 to 5:1, about 1:2 to 2:1, or about 1:1. Modification of the molar ratio allows control of the number of reactive groups per nanoparticle.

In an aspect, no or a very small amount (e.g., less than about 1% or less than about 2%) of primary amines can be detected on the surface of the coated magnetic nanoparticle using a standard assay. In one embodiment, the standard assay comprises 3-(4-carboxybenzoyl)quinoline-2-carboxaldehyde (CBQCA) or o-phthaldialdehyde (OPA). In one embodiment, the standard assay uses a CBQCA assay kit (Thermo Fisher).

The term “attached”, “bound”, “bond”, or “bonded” can include, but is not limited to, chemically bonded (e.g., covalently or ionically, directly or indirectly). In an embodiment, “bound”, “bond”, or “bonded” can include, but is not limited to, a covalent bond, a non-covalent bond, an ionic bond, a chelated bond, as well as being bound through interactions such as, but not limited to, hydrophobic interactions, hydrophilic interactions, charge-charge interactions, π-stacking interactions, combinations thereof, and like interactions.

In an aspect, the coated magnetic nanoparticle having the PEG-silanization coating can have one or more of the following characteristics. The coated magnetic nanoparticles have the same saturation magnetization as the original magnetic core, indicating that the PEG-silanization coating does not change or significantly change (e.g., less than 10%, less than 5%, less than 4%, less than 3%, less than 2%, or less than 1%) the magnetic properties of the original magnetic core. The coated magnetic nanoparticles are very stable in cryopreserved solution and do not aggregate over long periods of time (e.g., 10 days or more).

In an embodiment, the coated magnetic nanoparticle can have a diameter (or the longest dimension of the coated magnetic nanoparticle) of about 1 to 1000 nm, about 1 to 100 nm, about 1 to 30 nm, about 500 nm, about 100 nm, about 50 nm, about 30 nm, about 10 nm, or about 5 nm.

In an embodiment, the coating (e.g., excluding the magnetic core) can be about 1 to 5 nm thick, about 1 to 10 nm thick, about 1 to 20 nm thick, about 1 to 30 nm thick, about 1 to 40 nm thick, about 1 to 50 nm thick, about 1 to 60 nm thick, about 1 to 100 nm thick, about 1 to 200 nm thick, or about 1 to 1000 nm thick.

In an embodiment, a coating thickness of 2-3 nm is enough to provide a robust coating that will keep the magnetic nanoparticles stable (e.g., no aggregates or sediments formed) inside cryopreservation solution (e.g., VS55) for greater than 10 days, 20 days, 30 days, 40 days, or longer.

In one embodiment, the coated magnetic nanoparticle has a magnetic core, which can be generally spherical, semi-spherical, oval, or a similar three-dimensional shape. In an embodiment, the magnetic core can be made of a material such as iron oxide, magnetite, and substituted ferrite. In one embodiment, the substituted ferrite can be nickel ferrite, aluminum ferrite, manganese ferrite, zinc ferrite, cobalt ferrite, and combinations thereof. In one embodiment, the magnetic core is superparamagnetic. The magnetic core can have a longest dimension or diameter of about 2 nm to about 100 nm, about 5 nm to about 50 nm, about 5 nm to about 30 nm, about 5 nm to about 20 nm, about 3 nm to about 20 nm, or about 4 nm to about 20 nm. In an embodiment, if the magnetic core or nanoparticle is not spherical or semi-spherical, then the longest dimension of the magnetic core or nanoparticle is equivalent to the diameter and can have any one of the diameters (longest dimension) disclosed herein.

In one embodiment, the aminosilane can be of 3-aminopropyltrimethoxysilane, 3-aminopropylmethyldimethoxysilane, 3-aminopropyldimethylmethoxysilane, N-(2-aminoethyl)-3-aminopropylmethyldimethoxysilane, N-(2-aminoethyl-3-aminopropyl)trimethoxysilane, 4-aminobutyldimethylmethoxysilane, 4-aminobutyltrimethoxysilane, aminoethylaminomethylphenethyltrimethoxysilane, N-(2-aminoethyl)-3-aminoisobutylmethyldimethoxysilane,N-(6-aminohexyl)aminopropyltrimethoxysilane, 3-(m-aminophenoxy)propyltrimethoxysilane, aminophenyltrimethoxysilane, 3-aminopropyltriethoxysilane, 3-aminopropylmethyldiethoxysilane, 3-aminopropyldimethylethoxysilane, N-(2-aminoethyl)-3-aminopropylmethyldiethoxysilane, N-(2-aminoethyl-3-aminopropyl)triethoxysilane, 4-aminobutyldimethylethoxysilane, 4-aminobutyltriethoxysilane, aminoethylaminomethylphenethyl triethoxysilane, N-(2-aminoethyl)-3-aminoisobutylmethyldiethoxysilane, N-(6-aminohexyl)aminopropyltriethoxysilane, 3-(m-aminophenoxy)propyltriethoxysilane, aminophenyltriethoxysilane, and combinations thereof or derived there form. In another embodiment, the aminosilane is 3-aminopropyl triethoxysilane.

In one embodiment, the first PEG-silanization coating is derived from PEG-silane having an alkoxy group on a terminus of the PEG moiety opposite a terminus conjugated to a silane moiety.

In one embodiment, the first PEG-silanization coating is derived from PEG-silane having the formula:

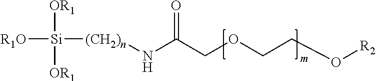

wherein R1 is H or an alkyl group such as C1-C6 alkyl in particular a methyl or ethyl group;
R2 is H or an alkyl group such as C1-C6 alkyl in particular a methyl or ethyl group;
n is 1, 2, 3, 4, 5, or 6;
m is an integer or about 10 to about 1000.

In one embodiment, the first PEG-silanization coating comprises the formula:

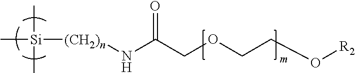

wherein R2 is H or an alkyl group such as C1-C6 alkyl in particular a methyl or ethyl group;
n is 1, 2, 3, 4, 5, or 6;
m is an integer.

In one embodiment, R2 is methyl, and n is 3. In one embodiment, R2 is methyl, and n is 3. In one embodiment, m is an integer from about 5 to about 1000, from about 10 to about 1000, from about 5 to 500, from about 5 to about 1000.

In one embodiment, the PEG-silane has molecular weight of from about 1 kDa to about 20 kDa, from about 2 kDa to about 10 kDa, from about 3 kDa to about 9 kDa, from about 4 kDa to about 8 kDa, or from about 5 kDa to about 7 kDa.

In an embodiment, the second PEG group has at least one amine reactive group, where the amine reactive group can react with an amino group on the aminosilane. The amine reactive group can be a group such as a carboxy group (e.g., —COOH) or a succinimidyl ester group. In addition, the second PEG group having at least one amine reactive group can also include one or more secondary PEG moieties attached to the PEG group, for example at the end of the PEG group opposite the amine reactive group. The secondary PEG moiety can be a thiol reactive group (e.g., a thiol group, a maleimide, or a 3-arylpropiolonitrile group), a click reactive group (e.g., azide, alkyne, norbornene, thiol, BCN (bicyclo[6.1.0]nonyne), DBCO ((Dibenzocyclooctyne)), a fluorophore, a peptide, a targeting agent (e.g., compounds that have an affinity towards a type of cell or tissue such as cancer cells or the like), a combination thereof (e.g., a secondary PEG moiety such as the thiol reactive group or the click reactive group that is bonded to a fluorophore, a peptide, or a targeting agent), and the like. In an aspect, the targeting agent can include: a protein, an antibody (monoclonal or polyclonal), an antigen, a polynucleotide, an enzyme, a hapten, a polysaccharide, a sugar, a fatty acid, a steroid, a glycoprotein, a carbohydrate, a lipid, a purine, a pyrimidine, an aptamer, a small molecules, a ligand, or combinations thereof. In as aspect, the secondary PEG moiety (e.g., one of the reactive groups) can be reacted with the targeting agent, for example, the secondary PEG moiety is a thiol and then a targeting agent is bonded to the thiol group. In one embodiment, the second PEG group can have a molecular weight of about 0.5 kDa to about 20 kDa, about 0.5 kDa to about 10 kDa, about 1 kDa to about 9 kDa, about 2 kDa to about 8 kDa, about 3 kDa to about 7 kDa, or about 4 kDa to about 4 kDa.

The term “affinity” can include biological interactions and/or chemical interactions. The biological interactions can include, but are not limited to, bonding or hybridization among one or more biological functional groups located on the biological target and/or the capture agent. The chemical interaction can include, but is not limited to, bonding among one or more functional groups (e.g., organic and/or inorganic functional groups) located on the capture agent and/or biological agent. In an aspect, the targeting agent has a strong preference (e.g., 90% or more, 95% or more, 99% or more, or 99.9% or more) to bond with the target of interest over other components that might be present so that the target agent is an effective way to sense and detect the presence of the targets in the samples of interest or subject.

Now having described the coated magnetic nanoparticle, additional details and uses will be described. In an embodiment, the coated magnetic nanoparticle can be used in imaging, where the coated magnetic nanoparticle can be used advantageously due to the long blood circulation lifetime. In general, an effective amount (e.g., an amount sufficient to achieve the desired imaging result (e.g., detectably effective amount)) a composition comprising the coated magnetic nanoparticle can be introduced (e.g., administered (e.g., oral, intravenous, and the like)) to a subject (e.g., a living human or mammal) or organs, tissues, or cells. After a period of time (e.g., minutes, an hour, or longer), the subject or organs, tissues, or cells is introduced to an imaging device (e.g., MRI) and the subject (or an area of the subject) or organs, tissues, or cells is imaged. In an aspect, the coated magnetic nanoparticle can include a targeting agent to detect the presence of a disease or the like, for example a type of cancer.

In an embodiment, the coated magnetic nanoparticle can be produced using the procedures described in detail in Example 1 and 2. In general, a magnetic core can be mixed with a first composition comprising a mixture of poly(ethylene glycol)-silane (PEG-silane) group and aminosilane to form a first nanoparticle coated with a first layer. The first layer can include a first ligand derived from the PEG-silane group. The first ligand is bonded (e.g., covalently attached) directly or indirectly (e.g., through the formation of a siloxane shell) to the magnetic core. The aminosilane is bonded (e.g., covalently attached) directly or indirectly (e.g., through the formation of a siloxane shell) to the magnetic core. Then the first nanoparticle is introduced to a second composition comprising a PEG group (also referred to as “second PEG group”) to form a second nanoparticle (e.g., the coated magnetic nanoparicle) further coated with a second layer comprising PEG group. The PEG group has at least one amine reactive group. The second PEG coating is attached to the first PEG-silanization coating via an amino group on the aminosilane and the amine reactive group on the second PEG coating. In one embodiment, the method further comprises coating the magnetic core with oleic acid ligand on the surface and replacing the oleic acid ligand with PEG-silane. In an aspect, second PEG groups can include a second PEG moiety such those described herein as well as combination thereof (e.g., one of the reactive groups with a fluorophore, peptide, or targeting agent). In an aspect, no or very little primary amines can be detected on a surface of the second nanoparticle using a standard assay. The product of this process is the coated magnetic nanoparticle as described above and herein, where the dimensions and other aspects described above and herein apply to the product formed. In this regard, the present disclosure includes compositions made from the methods described herein.

In another aspect, the present disclosure provides a cryopreservation composition including a cryopreservation agent, and the coated magnetic nanoparticles disclosed herein. In one embodiment, the cryopreservation agent can be of VS55, DP6, and glycerol. In one embodiment, cryopreservation agent is VS55.

TABLE A

| Composition | VS55 (8.4M) | | DP6 (6M) | |
|---|---|---|---|---|
| Dimethyl Sulfoxide (DMSO) | 3.1M | | 3M | |
| Propylene Glycol (PG) | 2.21M | | 3M | |
| Formamide | 3.1M | | — | |
| HEPES | 2.4 | g/L | 2.4 | g/L |
| D-Glucose | 0.194M | | 0.194M | |
| Potassium Phosphate | 0.015M | | 0.015M | |
| Monobasic (KH2PO4) | | | | |
| Potassium Phosphate | 0.042M | | 0.042M | |
| Dibasic (K2HPO4) | | | | |
| Potassium Chloride (KCl) | 0.015M | | 0.015M | |
| Sodium Bicarbonate (NaHCO3) | 0.010M | | 0.010M | |
| Critical Cooling Rate (CCR) | −2.5° | C./min | −40 | C./min |
| Critical Warming Rate (CWR) | 55 | C./min | 185 | C./min |

In one embodiment, the coated magnetic nanoparticles can be present in the cryopreservation composition in an amount of at least 0.01 mg of magnetic atoms per milliliter of the cryopreservation composition such as, for example, at least 1.0 mg/ml, at least 2.0 mg/ml, at least 3.0 mg/ml, at least 4.0 mg/ml, at least 5.0 mg/ml, at least 6.0 mg/ml, at least 7.0 mg/ml, at least 8.0 mg/ml, at least 9.0 mg/ml, at least 10 mg/ml, at least 11 mg/ml, at least 12 mg/ml, at least 13 mg/ml, at least 14 mg/ml, at least 15 mg/ml, at least 20 mg/ml, at least 25 mg/ml, or at least 50 mg/ml. In some embodiments, the coated magnetic nanoparticles can be present in the cryoprotective composition in an amount of no more than 100 mg/ml, no more than 75 mg/ml, no more than 50 mg/ml, no more than 25 mg/ml, no more than 20 mg/ml, no more than 15 mg/ml, no more than 10 mg/ml, no more than 9 mg/ml, no more than 8 mg/ml, no more than 7 mg/ml, no more than 6 mg/ml, or no more than 5 mg/ml.

The coated magnetic nanoparticles can be present in the cryopreservation composition in an amount sufficient to provide minimum at least 0.01 mg of magnetic atoms per milliliter of the vitrified biological material such as, for example, at least 1.0 mg/ml, at least 2.0 mg/ml, at least 3.0 mg/ml, at least 4.0 mg/ml, at least 5.0 mg/ml, at least 6.0 mg/ml, at least 7.0 mg/ml, at least 8.0 mg/ml, at least 9.0 mg/ml, at least 10 mg/ml, at least 11 mg/ml, at least 12 mg/ml, at least 13 mg/ml, at least 14 mg/ml, at least 15 mg/ml, at least 20 mg/ml, at least 25 mg/ml, or at least 50 mg/ml. In some embodiments, the coated magnetic nanoparticles can be present in the cryoprotective composition in an amount sufficient to provide a maximum of no more than 100 mg/ml, no more than 75 mg/ml, no more than 50 mg/ml, no more than 25 mg/ml, no more than 20 mg/ml, no more than 15 mg/ml, no more than 10 mg/ml, no more than 9 mg/ml, no more than 8 mg/ml, no more than 7 mg/ml, no more than 6 mg/ml, or no more than 5 mg/ml. In some embodiments, the amount of the coated magnetic nanoparticles in the cryoprotective composition may be characterized as a range having endpoints defined by any minimum amount listed above and any maximum amount listed above that is smaller than the maximum amount. In one embodiment, the magnetic atom is Fe.

In another aspect, the present disclosure provides a composition including a biomaterial perfused with the cryopreservation composition disclosed herein. In one embodiment, the biomaterial comprises an organ or portion thereof, a tissue or portion thereof, or cells.

In another aspect, the present disclosure provides a method including the steps of contacting a biomaterial with the composition disclosed herein; freezing the biomaterial; and rewarming the biomaterial. In one embodiment, the biomaterial is rewarmed at a rate of at least 100° C./min, 150° C./min, 200° C./min, 250° C./min, or 300° C./min. In one embodiment, rewarming the biomaterial includes subjecting the biomaterial to electromagnetic energy of an intensity, and for a duration, effective to thaw the biomaterial.

In some embodiments, the electromagnetic energy can include a radio frequency field, alternating magnetic field, or rotating magnetic field. In such embodiments, the electromagnetic energy can exhibit a minimum frequency of no more than 1 MHz such as, for example, no more than 750 Hz, no more than 500 Hz, no more than 375 Hz, no more than 300 Hz, no more than 250 Hz, no more than 225 Hz, no more than 200 Hz, no more than 175 Hz, no more than 150 Hz, no more than 125 Hz, no more than 100 Hz, no more than 75 Hz, or no more than 50 Hz. In some embodiments, the alternating magnetic field can exhibit a maximum frequency of at least 1 Hz such as, for example, at least 5 Hz, at least 10 Hz, at least 25 Hz, at least 50 Hz, at least 75 Hz, at least 100 Hz, at least 125 Hz, at least 150 Hz, at least 175 Hz, at least 200 Hz, at least 225 Hz, or at least 250 Hz. In some embodiments, the alternating magnetic field may be characterized by a range of frequencies having as endpoints any minimum frequency listed above and any maximum frequency listed above that is greater than the minimum frequency and may be time-dependent. In some embodiments, for example, the alternating magnetic field may have a frequency ranging from about 175 Hz to about 375 Hz. In another embodiment, the frequency may range from 200 Hz to about 300 Hz.

In some embodiments, the alternating magnetic field may have a minimum strength of at least 1 kA/m such as, for example, at least 5 kA/m, at least 10 kA/m, at least 20 kA/m, at least 30 kA/m, at least 40 kA/m, at least 50 kA/m, at least 75 kA/m, or at least 100 kA/m. In some embodiments, the alternating magnetic field may have a maximum strength of no more than 200 kA/m such as, for example, no more than 150 kA/m, no more than 100 kA/m, no more than 80 kA/m, no more than 60 kA/m, or no more than 50 kA/m. In some embodiments, the strength of the alternating magnetic field may be characterized as a range having as endpoints any minimum strength listed above and any maximum strength listed above that is greater than the minimum strength and may be time-dependent. In some embodiments, the alternating magnetic field may have a strength of from about 10 kA/m to about 100 kA/m. In one embodiment, the alternating magnetic field may have a strength of about 40 kA/m to about 50 kA/m.

### EXAMPLES

### Example 1

The present examples provide formulation of SPIONs coated with a dense, covalently grafted brush of poly (ethylene glycol) that are stable against aggregation in CPA solutions for prolonged periods and after vitrification and nanowarming from liquid nitrogen temperature to room temperature. The present examples demonstrate that this magnetic cryoprotecting agent (mCPA) possesses fast heating rates, controllable through the magnitude of the applied alternating magnetic field and SPION composition. The present examples further demonstrate that these mCPAs can uniformly perfuse whole rat hearts and be efficiently removed after vitrification and nanowarming. For this purpose, the present examples demonstrate the application of magnetic particle imaging (MPI) to quantitatively assess SPIO loading in the organ before and after vitrification and nanowarming. MPI quantifies the spatial 3-dimensional distribution of iron oxide nanoparticle tracers (24, 25). The image signal in each voxel is proportional to the concentration of the particles, and there is negligible signal attenuation by tissue. Together, these examples suggest the potential of the SPIONs and mCPA solutions disclosed here for whole-heart cryopreservation and nanowarming.

### Superparamagnetic Iron Oxide Nanoparticles (SPION) Synthesis

SPIONs were synthesized by a co-precipitation method optimized to produce particles with high-energy dissipation rates, described by others (32). Deionized water was de-oxygenated for 30 minutes by bubbling nitrogen. Then, 3.98 g of iron(II) chloride tetrahydrate (99%, Sigma-Aldrich), and 10.81 g iron(III) chloride hexahydrate (99%, Sigma-Aldrich) was dissolved in 100 mL of the de-oxygenated deionized water. Once the iron salt solutions dissolved, each solution was de-oxygenated for 5 minutes and mixed in a glass reactor. The reaction mixture was heated to 75° C., and approximately 35 mL of ammonium hydroxide (29% v/v, Fisher Scientific) was added to the mixture quickly, the pH should have reached 8.0-8.5. The reaction temperature was then increased to 85° C. The synthesis was conducted for one hour while maintaining a pH of around 8.0-8.5 by periodic addition of ammonium hydroxide. The resulting SPIONs were centrifuged, and the supernatant discarded.

The black colloid from synthesis was suspended in tetramethylammonium hydroxide (TMAOH, 1 M, Sigma-Aldrich) at a volume ratio of 1:2 SPION/TMAOH. The peptization process was performed twice using an ultra sonicator (Q700, Qsonica Sonicators) for 30 minutes each time. The suspension was centrifuged again, and the peptized SPIONs were resuspended in water.

Oleic acid (OA, 90%, Sigma-Aldrich) adsorption onto the nanoparticles facilitates coating the SPIONs with polyethylene glycol (PEG). 15 g OA/g SPION was added to the SPION solution and ultrasonicated (Q700, Qsonica Sonicators) for 15 min. The mixture was transferred to a glass reactor where it was heated to 50° C. and held at temperature to react for 2 h. Precipitation of the SPIONs was performed using twice the volume of ethanol (200 proof, Decon Laboratories) and magnetically decanted to separate the particles, which were finally suspended in toluene (>98%, Sigma-Aldrich).

SPION Coating with Polyethylene Glycol

A two-step process was used to synthesize PEGsilane. First, 5 kDa molecular weight monomethoxy PEG (mPEG, 99.999%, Sigma-Aldrich) was converted to mPEG-COOH as described by Lele et al. (33) Briefly, 50 g of mPEG was dissolved in 400 mL of acetone (99.8%, Fisher Chemicals). Jones reagent, a strong oxidizing agent comprised of chromium trioxide in aqueous sulfuric acid, was used to oxidize mPEG. Once the mPEG was dissolved in acetone, 16.1 mL of Jones reagent was added and reacted for 24 hours. Excess isopropyl alcohol (70%, Sigma-Aldrich) was added to stop the reaction. Activated charcoal (12-40 mesh, ACROS Organics) was used to remove impurities from the reaction. Activated charcoal and chromium salts were removed by vacuum filtration. Then, the acetone solution containing the oxidized mPEG was concentrated using a rotary evaporator. The concentrated mixture of mPEG-COOH was re-dissolved in 1 M hydrochloric acid (37% w/v, Fisher Chemicals). The polymer was extracted to the organic phase by liquid-liquid extraction using approximately 75-100 mL of dichloromethane (>99.5%, Sigma-Aldrich). The process was performed twice, and all of the solution was concentrated by rotary evaporation. Finally, the mPEG-COOH was precipitated using cold diethyl ether (>99.8%, Fisher Chemicals). The mPEG-COOH was then dried in a vacuum oven at room temperature.

PEGsilane was obtained by performing amidation of mPEG-COOH with 3-aminopropyl triethoxysilane (APS, TCI America). Briefly, mPEG-COOH was weighed and melted in an oil bath set to 60° C. Then, APS was added to the melted PEG at a 1:1 molar ratio of mPEG-COOH/APS. The mixture was allowed to react for 2 hours at 120° C. and 500 mbar. The PEGsilane was then cooled to room temperature and hardened.

The SPIONs were coated with PEGsilane using ligand exchange, replacing the oleic acid on the surface of the nanoparticles with PEGsilane, following a previously described procedure (27). Briefly, 3.5 g of PEGsilane was dissolved in 250 mL dry toluene. A 45° C. water bath was used to dissolve the PEGsilane in toluene and when dissolved, 250 mL of OA adsorbed SPIONs at 0.8 mg/mL and 40 μL of acetic acid (99.8%, ACROS Organics) were added and mixed. Acetic acid was used to catalyze hydrolysis and condensation of siloxane groups onto the SPION surface. The solution was then placed in a shaker for 72 hours. Cold diethyl ether was added to precipitate the nanoparticles to recover the PEGsilane coated SPIONs. The precipitate was then dried in a vacuum oven at room temperature overnight. The next day, PEGsilane coated SPIONs were resuspended in water and dialyzed to remove excess PEGsilane. For further purification, particles were purified using magnetic columns (Miltenyi Biotec).

Particles were stabilized by backfilling them with additional oxidized PEG using EDC-NHS chemistry. The number of remaining primary amines on the particles was quantified using a CBQCA assay kit (Thermo Fisher), following the manufacturer's protocol. Once the number of amines was determined, a ratio of 1:10 amine to carboxylic acid was used. The mPEG-COOH was suspended in water and pH adjusted to 5.0. A 1:2 ratio of carboxylic to EDC (Thermo Fisher) was added and allowed to react for 15 minutes to activate the carboxylic group. Then, sulfo-NHS (Thermo Fisher) was added at a 1:1 ratio of EDC to sulfo-NHS. The pH of the solution was slowly adjusted to 8.0 and the solution of particles was added once the pH was reached. The mixture reacted overnight and was then purified using a magnetic column as described above.

### Magnetic Cryoprotecting Agent Solution (Mcpa) Formulation

VS55 is an 8.4 M cryopreservation solution including 2.2 M propylene glycol (Fisher Chemicals), 3.1 M formamide (Fisher BioReagents), 3.1 M dimethyl sulfoxide (Fisher BioReagents), and 10 mM of HEPES (Fisher BioReagents) in Euro-Collins solution (16, 29). Euro-Collins is composed of 194 mM D-glucose (Fisher BioReagents), 15 mM potassium phosphate monobasic (Fisher BioReagents), 42 mM potassium phosphate dibasic (Fisher BioReagents), 15 mM potassium chloride (Fisher BioReagents), and 10 mM sodium bicarbonate (Sigma-Aldrich) (29). 200 mL of a 5× concentrated Euro-Collins solution was mixed with 2.39 g of HEPES, 139.56 g of formamide, and 168.38 g of propylene glycol to make 1L of VS55. The solution was mixed well before adding 242.14 g of dimethyl sulfoxide. Last, deionized water was added to complete to 1 L. It was filtered through a 0.2 micron nylon filter before any in vitro or ex vivo experiments to sterilize the solution.

A stock solution of mCPA containing 10 mg Fe per mL was produced by following the VS55 preparation procedure, but instead of adding deionized water at the end to complete to 1 L, a solution of stable PEG-coated SPIONs suspended in deionized water at a concentration of 35 mg_{Fe}/mL was added. The solution was sterilized by filtering through a 0.2 micron nylon filter before any in vitro or ex vivo experiments.

Characterization of VS55, SPIONs, and mCPA

Dynamic light scattering (DLS). The hydrodynamic diameter of the particles was obtained by dynamic light scattering (DLS) using a Brookhaven Instruments 90Plus/BI-MAS operating at room temperature. All measurements were made at a scattering angle of 90°.

Transmission electron microscopy (TEM). Samples were prepared for electron microscopy by depositing a drop of the nanoparticles in solution at 1 mg/mL on a formvar-coated copper grid. A JEOL 200CX microscope operated at 120 kV (Peabody, MA. USA) was used to obtain images. The number-weighted mean diameter and the geometric deviation were obtained by fitting the data to a lognormal size distribution.

Magnetic measurements. Equilibrium magnetic measurements were performed using a Quantum Design MPMS-3 Superconducting Quantum Interference Device (SQUID) magnetometer. Magnetization curves were obtained for dry SPIONs or 20 μd of mCPA at 10 mg_{Fe}/mL samples at 77.15K and 300 K in a magnetic field range of 7 to−7 T. From these curves, the experimental remanence and coercivity was determined.

### Evaluation of Nanowarming

Nanowarming of the mCPA solution was evaluated using 20 mL of solution in a 32 mm diameter specimen jar. A fiber optic temperature probe (Qualitrol) was placed in the middle of the sample to record the temperature. The sample was vitrified using a mechanical freezer by setting the freezer to cool at 15° C./min. Heating was performed by applying an alternating magnetic field (AMF) using an Ambrell EasyHeat induction heater. A control solution of VS55 was compared with an mCPA solution at 10 mg_{Fe}/mL to assess nanowarming. The warming of both solutions was evaluated by immersion in a water bath set at 37° C. and separately by applying AMFs (42.5 kA/m peak, 278 kHz). The field amplitude and concentration of particles were varied separately to evaluate the control of nanowarming. The field strengths tested were 42.5 kA/m, 30.6 kA/m, and 17.2 kA/m peak at a frequency of 278 kHz. The concentrations used were 10 mg_{Fe}/mL, 5 mg_{Fe}/mL, 2.5 mg_{Fe}/mL, and 1 mg_{Fe}/mL.

### SPION Colloidal Stability

Colloidal stability was assessed using dynamic light scattering (DLS). SPIONs were suspended in PBS 1× or VS55 at approximately 0.5 mg_{Fe}/mL. Colloidal stability was assessed for a month. Most importantly, stability before vitrification and after vitrification/nanowarming was assessed. A stock of 20 mL mCPA at 10 mg_{Fe}/mL sample was made, at day 10, a 100 μL aliquot from the mCPA was obtained and diluted into 2 mL of VS55 for DLS measurements. The sample was then vitrified and nanowarmed. Once the sample was rewarmed, another aliquot of 100 μL was obtained and diluted for DLS measurement. The colloidal stability was studied for one month.

Effect of SPIONs and mCPA on Primary Cardiomyocyte Viability In Vitro

All of the following studies were done using research protocols approved by the Institutional Animal Care and Use Committee (IACUC) at the University of Florida. Primary cardiomyocytes from neonatal rat hearts were used in conjunction with Hoechst-PI assay to evaluate cell viability in the presence of CPA at different dilutions (25%, 50%, 75%, and 100%), SPIONs at different concentrations (1, 2.5, 5, and 10 mg_{Fe}/mL), and mCPA at 5 mg_{Fe}/mL added gradually.

Pierce Primary Cardiomyocyte Isolation Kit (Thermo Fisher) was used to isolate cardiomyocytes. Neonatal hearts were obtained from 2-3 day old rats, within 30 minutes of the rat being euthanized. Cardiomyocytes were isolated and grown following the protocol provided in the isolation kit. Isolated cardiomyocytes were cultured in a 48 well plate at a density of 400,000 cells/well and allowed to grow for three days before performing the experiments.

All experiments were performed on ice in a cold room at 4° C. to provide a temperature-controlled environment in order to minimize the toxicity of VS55. Cells were cooled for 10 minutes before beginning experiments. The solution was added and allowed to incubate for 21 minutes before removal (chosen based on typical times reported for CPA perfusion in organs in the literature) (15, 17, 28, 29, 34), incubated with Hoechst-PI for 5 minutes, and visualized under the microscope to assess VS55 and SPION toxicity. Hoechst stains all nuclei in blue and PI stains all apoptotic/necrotic cells in red. The percentage of live cells was determined by calculating the difference between dead cells and the total number of cells.

Loading and removal of VS55 and mCPA solutions were done in 3-minute steps. gradually increasing the concentration as follows: 12.5% VS55→25% VS55→50% VS55→75% VS55→100% mCPA→50% VS55→12.5% VS55 →media. The cells were then incubated with Hoechst-PI for 5 minutes and visualized under the microscope. Viability was reported as the percentage of live to total cells counted from multiple images (over 6000 cells per group), where n=3 for each condition.

Evaluating Whole Heart Perfusion and Removal with mCPA

Male Sprague Dawley rats, four months old, were used for these studies. Initially, perfusion of whole rat hearts with mCPA was evaluated without vitrification or nanowarming. The approach was to (1) remove the heart; (2) perfuse it with Custodiol® HTK; (3) perfuse it with mCPA; (4) image it using MPI; (5) perfuse the heart with Custodiol® HTK to remove the mCPA; and (6) image the heart using MPI. To remove both the heart and lungs, the rat was first anesthetized and injected with 500 USP units of heparin to avoid clotting. Three minutes after administering heparin, the thoracic cavity was opened, and the following were ligated: inferior vena cava, superior vena cava, brachiocephalic trunk, left common carotid artery, and the left subclavian artery. Once these were tied, the aorta and the inferior vena cava (below the tie) were cut, and the heart and lungs were removed and placed on ice. A plastic 26G needle was tied in the aorta. A syringe filled with Custodiol® HTK solution was attached to the needle and the heart and lungs were perfused with 7 mL to remove the blood. The superior vena cava ligature was removed for the perfusate to exit. The syringe was replaced with a syringe filled with mCPA at 1 mg_{Fe}/mL and 1.5 mL was injected. An additional 6 mL of Custodiol® HTK solution was perfused as described above to perfuse out the particles.

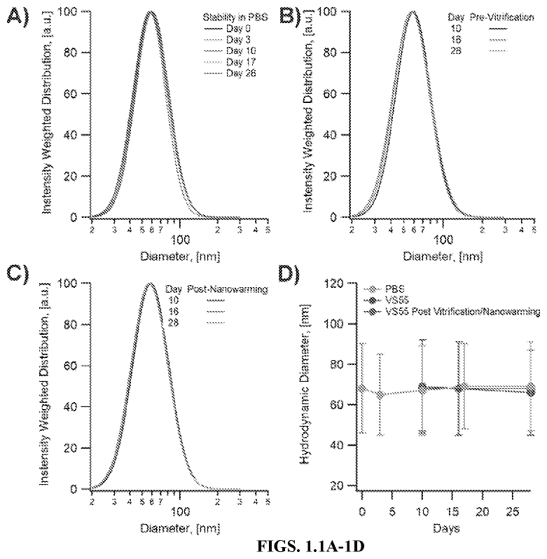

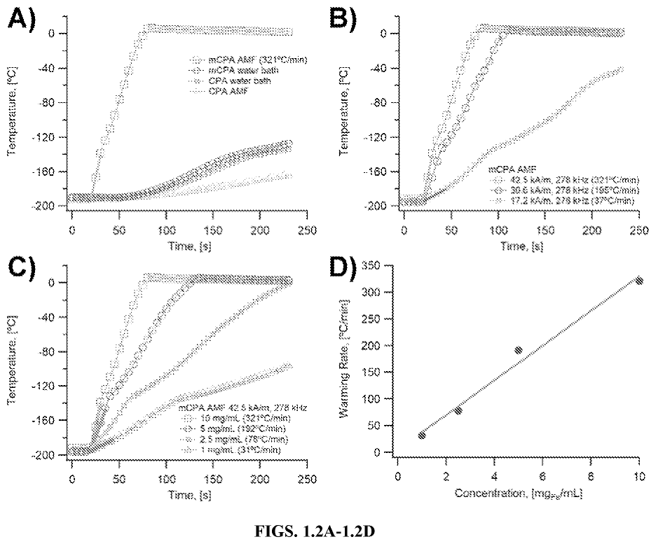

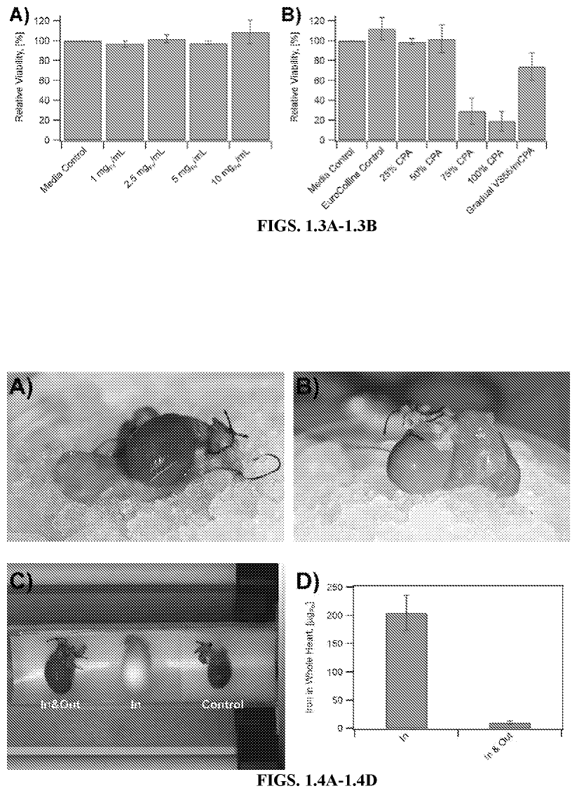

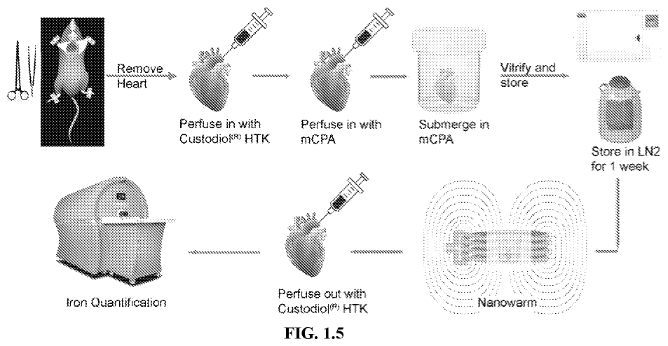

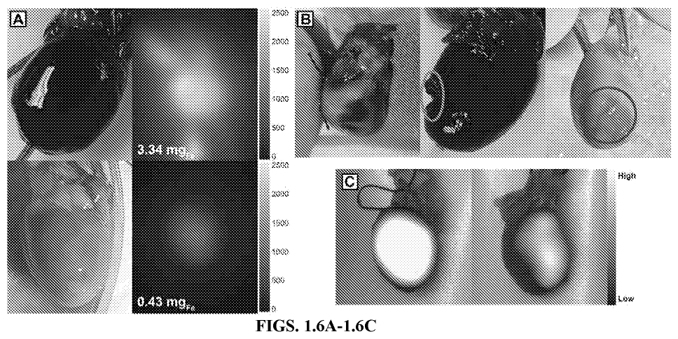

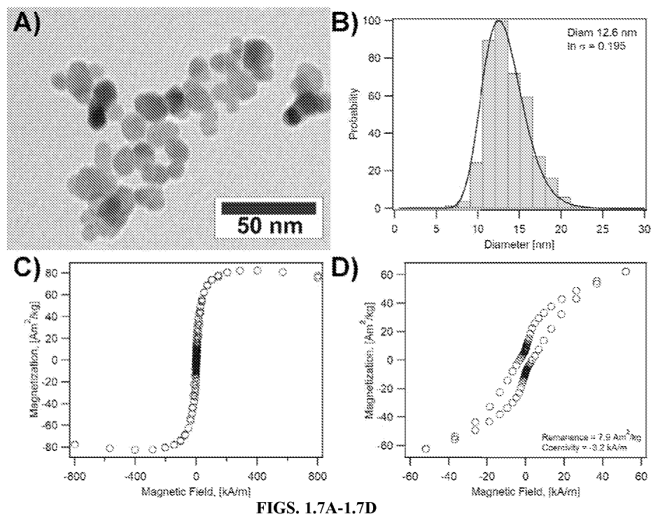

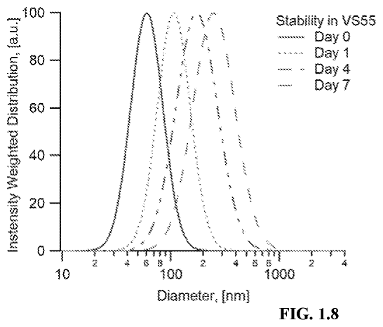

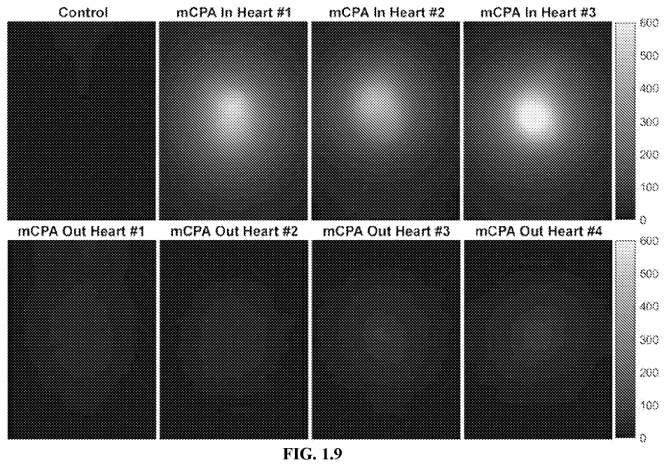

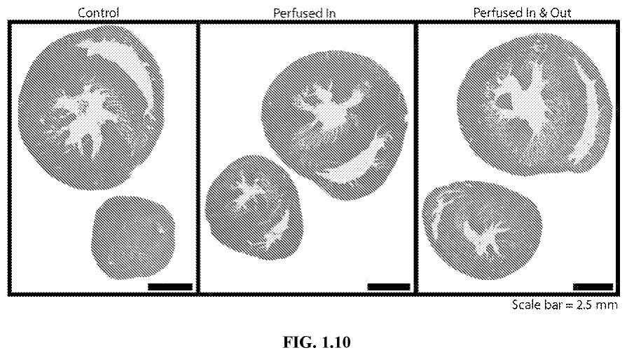

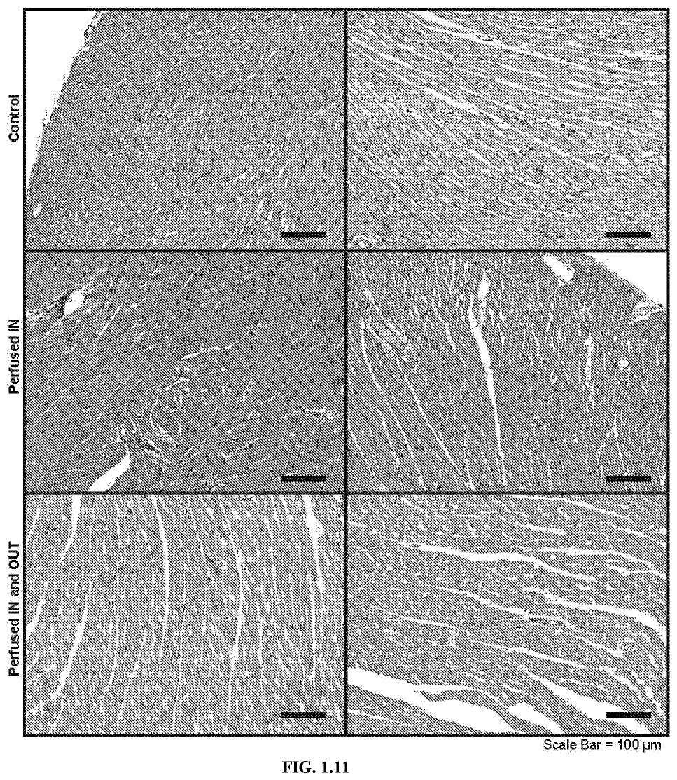

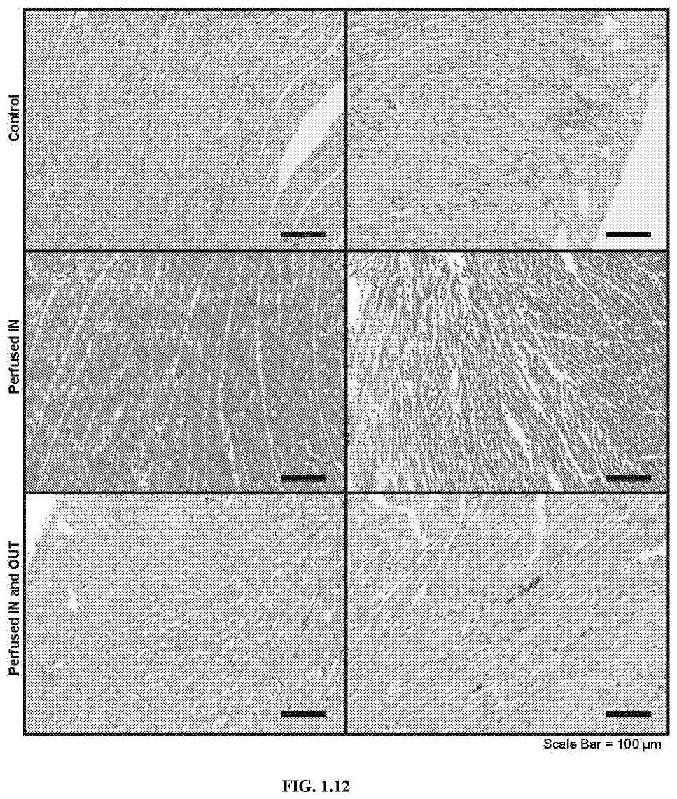

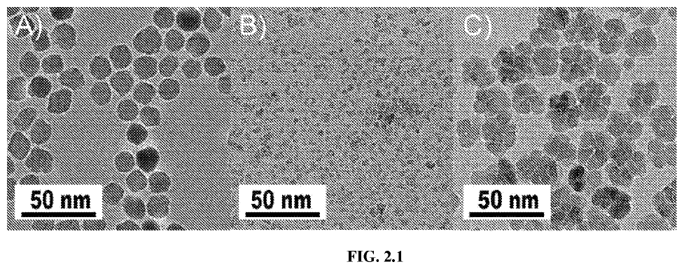

Perfusion of whole rat hearts with mCPA was evaluated, followed by vitrification, nanowarming, and removal of the mCPA solution. FIG. 1.5 illustrates the procedure taken for these experiments. The procedures to remove the heart and perfuse the organ were similar to those described above. The mCPA used in these experiments had a SPION concentration of 5 mg_{Fe}/mL, needed to achieve the required CWR of greater than 50° C./min. Once hearts were perfused with mCPA, a 20G plastic needle was placed in the heart's center to insert the fiber optic temperature probe used to monitor temperature during nanowarming. The heart was then submerged in the same mCPA solution used during perfusion and placed in a mechanical freezer to vitrify at a cooling rate of 15° C./min down to liquid nitrogen temperature of 196° C. Once the heart was vitrified, it was stored in a dewar filled with liquid nitrogen. One week later, the heart was removed from storage and nanowarmed using a field strength of 42.5 kA/m peak at a frequency of 278 kHz. Once the temperature inside the heart reached 0° C., the field was turned off, and the mCPA perfused out with Custodiol® HTK solution.

A MOMENTUM™ imager (Magnetic Insight, Inc.) was used to evaluate SPION perfusion through MPI. The hearts were imaged using 2D maximum intensity projection scans with a 12 cm×6 cm field of view in isotropic mode at 5.9 T/m. A fiducial of known SPION concentration was imaged along with the heart for quantification. Images were analyzed using VivoQuant by selecting the heart and calculating the total signal observed in the region of interest, compared to the total signal obtained from the fiducial.

Hearts were fixed in formalin and sent to the UF College of Medicine Pathology Core for sectioning and staining. Transversal cross-sections from the center and bottom of the rat hearts were obtained. Sections were 4 μm thick. Sections were stained using hematoxylin and eosin (H&E) and Prussian blue. H&E staining was used to assess heart structure, while Prussian blue was used as a complementary method to visualize particles in the heart.

SPIONs that are Stable in VS55 Before and After Vitrification and Nanowarming

SPIONs were synthesized by the coprecipitation method. The size distribution of the iron oxide cores, obtained by transmission electron microscopy, was fitted to lognormal size distribution, resulting in a number weighted mean core diameter of 12.6 nm and geometric deviation of 0.195 (FIGS. 1.7A-1.7D). The nanoparticles displayed superparamagnetic behavior (FIGS. 1.7A-1.7D), with small coercivity and remanence, and with a saturation magnetization of 82.4 Am²/kg, close to the bulk value for magnetite of 86.6 Am²/kg (26).

The colloidal stability of nanoparticles developed for biomedical applications is usually tested in saline solution or media. Due to the complexity of cryopreservation solutions, which often contain high concentrations of dimethylsulfoxide (DMSO) and other chemicals, it is important to test the stability of nanoparticles in these solutions when formulating a mCPA. SPIONs ligand exchanged with PEGsilane using previously reported procedures (27) were purified with dialysis, and magnetic separation to remove free PEGsilane. These nanoparticles were found to be unstable in VS55, aggregating quickly over a week (see FIG. 1.8). These nanoparticles' poor colloidal stability in VS55 may be caused by unreacted amines resulting from 3-aminopropyl triethoxysilane (APS) remaining in the PEGsilane (27). This results in a co-ligand exchange of PEGsilane and APS. However, this also suggests that the resulting particles have primary amines on the surface that allow “backfilling” with additional PEG. This resulted in SPIONs with excellent colloidal stability in VS55, as observed in FIG. 1.1. SPIONs coated and backfilled with PEG were stable against aggregation in VS55 for 28 days. The next step was to verify that the nanoparticles remain stable in VS55 and after the sample is vitrified and nanowarmed in an AMF. SPIONs were suspended in VS55 for 10 days, then were vitrified and nanowarmed in an AMF. Stability was assessed immediately after nanowarming and periodically over the next 18 days. FIG. 1.1C demonstrates the particles are stable post-vitrification. Importantly, FIG. 1.1D demonstrates that backfilled particles are the same size in PBS, VS55 pre- and post-nanowarming, and are stable against aggregation for at least 28 days.

Magnetic Cryopreservation Agent Solution with Fast and Controllable Nanowarming Rates

Nanowarming rates in an AMF were evaluated for CPA and mCPA (10 mg_{Fe}/mL) solutions and compared to those achievable by immersion in a water bath (FIG. 1.2A). Under AMF (42.5 kA/m, 278 kHz), the maximum heating rate achieved in a vitrified mCPA sample of 20 mL was 321° C./min, far surpassing the critical warming rate for VS55 of 50° C./min (14). In contrast, a VS55 sample treated under the same conditions did not rewarm quickly. Similarly, vitrified mCPA and VS55 solutions placed in a warm water bath (37° C.) did not rewarm quickly.

For mCPA at 10 mg_{Fe}/mL under an AMF, the solution heats up quickly and control of heating rate is desirable to avoid overshooting of target temperature during rewarming since chemical toxicity of cryopreservation agent solutions increases with temperature. Control of heating rate was achieved by adjusting the strength of the applied AMF. FIG. 1.2B demonstrates that the heating rate can be altered by controlling field strength. By holding frequency constant and changing the current in the coil, the field strength is adjusted. The results demonstrate that as field strength decreased, the heating rate decreased.

Another method to control the heating rate is through the concentration of backfilled PEG-coated SPIONs present in the mCPA. The concentration was varied from 1 mg_{Fe}/mL to 10 mg_{Fe}/mL. FIG. 1.2C demonstrates that the heating rate decreases with decreasing concentration at a fixed alternating magnetic field of 42.5 kA/m and 278 kHz. From the warming rates obtained at different concentrations, FIG. 1.2D shows that there is a linear relationship between the warming rate and SPION concentration. While these results show the potential to control heating rates through SPION concentration, they also suggest the importance of achieving uniform SPION loading throughout the organ to achieve uniform heating rates. This result underscores the need for methods to quantify SPION distribution in vitrified organs non-invasively.

Magnetic Cryopreservation Agent Solutions with Low Cardiomyocyte Toxicity

Toxicity of SPIONs and VS55 were evaluated in vitro to determine if the formulated mCPA is viable for future studies. Because the organ of interest was the heart, we evaluated cytotoxicity in primary cardiomyocytes. Primary cardiomyocytes were isolated from 2-3 days old rat hearts. First, the toxicity of backfilled PEG-coated SPIONs was tested in the cells. FIG. 1.3A shows the percentage of viable cells for each group from the image analysis of the Hoechst-PI stain. From the results, even at the highest SPION concentration tested of 10 mg_{Fe}/mL, SPIONs do not appear cytotoxic to the cells.

Next, the cytotoxicity of VS55 on primary cardiomyocytes was evaluated. This evaluation shows that VS55 is slightly toxic to human dermal fibroblasts (17), but toxicity to primary cardiomyocytes is not reported. The cytotoxicity of VS55 at different concentrations was tested by exchanging cell culture media with a VS55/media mixture. Cytotoxicity was also tested when the concentration of VS55 was gradually increased in steps up to 100% VS55 and replaced with mCPA containing SPIONs, to mimic typical CPA perfusion protocols (17, 28, 29). All experiments were performed on ice in a cold room at 4° C. to provide a temperature-controlled environment and minimize the chemical toxicity of VS55. VS55 is composed of 3.1 M DMSO, which is toxic to cells, and chemical toxicity increases in a temperature-dependent manner (15). The results in FIG. 1.3B show that replacing media directly with VS55/media mixtures containing more than 50% VS55 results in significant cytotoxicity towards primary cardiomyocytes. However, gradually increasing the concentration of VS55 to 100%, followed by replacement with mCPA, resulted in 74% primary cardiomyocyte viability.

Whole Heart Perfusion with mCPA Assessed Using Magnetic Particle Imaging

Past studies on nanowarming of biologics with mCPA that have been performed with mCPA have used cells or small sections of tissue submerged in mCPA solution (17, 19, 20), or have reported nonuniform distribution of SPIONs for organs (liver, kidney, ovary) and hindlimbs perfused with mCPA (21). The mCPA reported here can perfuse a whole rat heart and can be removed afterward. For these ex vivo studies, both hearts and lungs were removed for perfusion before removing the heart for further analysis. The reason to remove both heart and lungs for the experiments was the difficulty of ligating small pulmonary arteries and veins in a rat without cutting other parts of the heart. This will be less of a problem for experiments with larger subjects.

To evaluate whether perfusion can be performed, the rat's heart and lungs were removed, perfused with Custodiol® HTK first to remove the blood and arrest the heart, and then perfused with 100% mCPA. This was performed for 3 subjects to verify that the loading of particles was successful. FIG. 1.4A is a representative photo of a heart perfused with mCPA, showing the heart turns brown due to the uniformly distributed SPIONs. All 3 hearts were then removed from the lungs and placed in the MOMENTUM™ imager for quantitative imaging.

Once it was determined that the heart could be perfused with mCPA, the next step was to remove the mCPA. Using additional hearts perfused with mCPA, the mCPA was perfused out of the heart using Custodiol® HTK. FIG. 1.4B is a representative photo of a heart after perfusing out the mCPA, showing the heart color is now pink because most of the particles have been removed. This was performed in 4 hearts to verify that unloading of particles was reproducible. These hearts were then placed in the MOMENTUM™ imager for quantitative imaging.

FIG. 1.4C is a representative co-registered image of one heart from each group and the signal from MPI. The co-registered image for the heart perfused in shows that the SPIONs were well distributed throughout the heart. The signal intensity of the particles from the MPI is proportional to the concentration of the particles. It can be observed from FIG. 1.6C that the heart perfused with mCPA shows a bright signal, whereas the heart perfused in and out shows a faint signal. There is no signal from the control heart. Individual MPI images for the hearts in each group can be found in the Supplementary Information (FIG. 1.9). These results show a 95% reduction in SPION mass from the hearts after perfusing out with Custodiol® HTK, suggesting effective removal of the mCPA, as shown in FIG. 1.4D.

Histological analysis was performed for all hearts, and representative images of whole hearts from each group can be found in FIGS. 1.10 to 1.12. H&E stains showed that the myocardium's cytoarchitecture is similar across groups, suggesting no gross macroscopic damage to the heart tissue. The use of Prussian blue, a complementary technique to visualize iron, further demonstrated that the SPIONs were perfused in and out of the heart. Cross-sections of hearts perfused in with mCPA showed blue stains throughout the heart in the interstitial space while the blue stain could no longer be observed in hearts perfused in and out.

Demonstration of Whole-Heart mCPA Perfusion, Vitrification, Cryostorage, and Nanowarming

Next, whether mCPA can be perfused out after heart vitrification, cryostorage, and nanowarming was evaluated. FIG. 1.5 illustrates the procedure for these experiments. FIG. 1.6A is an example of a heart successfully perfused with mCPA, vitrified to liquid nitrogen temperature, cryostored in liquid nitrogen for one week, nanowarmed in an AMF, and perfused out with Custodiol® HTK. The heart looks intact visually, and the mCPA was successfully removed after nanowarming with Custodiol® HTK, as indicated by the uniform pink color of the heart. Using MPI, it was determined that about 90% of the SPIONs were removed.

While FIG. 1.6A demonstrates successful mCPA perfusion and removal after vitrification, cryostorage, and nanowarming, we note that there were also several failed attempts at perfusing and removing the mCPA, some of which are represented in FIG. 1.6B. Examples of reasons for failure to perfuse in or out with mCPA included problems with air bubbles being entrained during perfusion, cracking of the heart during the nanowarming step, and problems caused by not fully submerging the heart in mCPA solution before vitrification. While these failed attempts underscore the need for further technique development, the results of FIG. 1.6A clearly show the potential to uniformly perfuse hearts with mCPA and then remove the SPIONs after vitrification, biobanking at liquid nitrogen temperature, and nanowarming.

As noted previously, it is critical to uniformly perfuse the organ with mCPA and achieve uniform SPION distribution to obtain uniform heating during the nanowarming step. As such, techniques that allow non-invasive quantitative evaluation of SPION distribution in the heart are of great interest. MPI can be used to quantify and assess the distribution of SPIONs in hearts. This is illustrated in FIG. 1.6C, which shows optical and MPI images of a heart that was well perfused with mCPA and a heart that was poorly perfused due to obstruction of the right ventricle with an air bubble. Because MPI can provide a 3D quantitative and tomographic view of the distribution of SPIONs in whole organs, these results suggest the potential of MPI for evaluation of successful mCPA perfusion before vitrification. Furthermore, MPI could be coupled with the ability to control the location of SPION heating (30), which would enable control of the resulting temperature distribution during nanowarming.

### DISCUSSION

Particles coated for biomedical applications are usually tested for stability in saline solution and media only. However, due to the complexity of cryopreservation solutions, which often contain high concentrations of DMSO and other chemicals, it is important to test the particles' stability when formulating a mCPA. The present disclosure has demonstrated that particles coated with PEG are initially not colloidally stable in VS55. However, once backfilled with more PEG, SPIONs were stable in VS55 for at least one month.

Nanoparticle solutions often risk irreversible aggregation when freezing unless stabilizing agents are used (22, 23). The present disclosure demonstrated the stability of SPIONs in CPA after vitrification and rewarming without stabilizing agents, as these can affect the behavior of CPAs. The backfilled PEG-coated SPIONs in VS55 were stable for at least a month in VS55, and the particles were the same size as in PBS post-nanowarming, indicating no aggregation occurred. These results suggest that the formulated mCPA is stable and can potentially perfuse in and out of whole organs after vitrification and rewarming.

Nanowarming is only effective for cryopreservation if the critical warming rate (CWR) of the cryopreservation agent can be achieved volumetrically. The formulated mCPA including stable SPIONs in VS55 achieved an exceptionally high-temperature rise rate of up to 321° C./min under an AMF with amplitude 42.5 kA/m and frequency 278 kHz, far exceeding the required CWR of VS55 (50° C./min). The temperature rise was controllable by altering the field amplitude or changing the nanoparticles' concentration in VS55. Furthermore, this suggests that the SPIONs disclosed here could be used to formulate cryopreservation solutions with lower chemical toxicity but concomitant higher CWR requirements, such as DP6, which requires a minimum heating rate of 185° C./min (17).

Loading of both CPA and SPIONs into whole organs with minimal toxicity is required for biobanking by vitrification to be translatable. The cytotoxicity of VS55 and the formulated mCPA in vitro was evaluated. Cardiomyocytes loaded directly with SPIONs at up to 10 mg_{Fe}/mL had no change in viability. In 1999, Taylor et al. introduced a multistep protocol for loading and unloading CPA as a way to reduce toxicity from CPA solutions (28). The literature shows that VS55 can be loaded into tissues and organs without significant toxicity using multistep protocols at low temperatures (10, 15, 28, 29, 31). Osmotic effects are reduced by this step-wise increase in cryoprotective agent concentration, while the rapid transfer and low-temperature help prevent damage by chemical toxicity. Following this multistep protocol with the addition of SPIONs at the end of VS55 loading, negligible toxicity was observed in the cardiomyocytes (FIG. 1.4), compared to not using the multistep protocol.

Finally, the ability to perfuse in and out of whole organs was demonstrated using the novel biomedical imaging technology MPI in whole hearts from rats. The present disclosure demonstrated, through photography, MPI, and histology, that it is feasible to perfuse whole rat hearts with mCPA and perfuse out at least 95% of all SPIONs that were perfused into whole rat hearts (FIG. 1.6). It is important to assess and quantify the distribution of the nanoparticles in the heart to determine that uniform nanowarming can be achieved. Using MPI, it is possible to quantify SPION loading and removal in whole rat hearts, and assess mCPA distribution throughout the heart, comparing cases with successful and unsuccessful perfusion (FIGS. 1.5 and 1.6). Further, the present disclosure demonstrated successful whole-heart perfusion with mCPA, vitrification to liquid nitrogen temperature, cryostorage in liquid nitrogen for one week, nanowarming in AMF, and removal of SPIONs, and illustrated examples of unsuccessful perfusion that suggest improvements needed to achieve uniform mCPA perfusion and nanowarming.

The magnetic cryopreservation agent solutions disclosed here, including superparamagnetic iron oxide nanoparticles especially formulated to be colloidally stable in the cryopreservation agent VS55, have low toxicity to primary cardiomyocytes, can achieve exceptionally high heating rates from liquid nitrogen temperature to room temperature, and can uniformly perfuse and be removed from whole hearts. The present disclosure demonstrates whole-heart perfusion, vitrification to liquid nitrogen temperature, cryostorage in liquid nitrogen for one week, nanowarming in an alternating magnetic field from liquid nitrogen temperature to room temperature, and removal of the iron oxide nanoparticles through a combination of optical imaging and magnetic particle imaging. The present disclosure supports the potential of nanowarming using magnetic cryopreservation agent solutions to change current organ preservation paradigms and greatly enhance the availability of viable donor organs for transplantation.

### EXAMPLE 1 REFERENCES

1. WHO-ONT, “Organ donation and transplantation activities,” (Global Observatory on Donation and Transplantation, 2016).
2. S. Giwa et al., The promise of organ and tissue preservation to transform medicine. Nature Biotechnology 35, 530 (2017).
3. J. K. Lewis et al., The grand challenges of organ banking: proceedings from the first global summit on complex tissue cryopreservation. Cryobiology 72, 169-182 (2016).
4. T. D. o. Transplantation. (2019), vol. 2019.
5. P. E. Morrissey, M. L. Flynn, S. Lin, Medication noncompliance and its implications in transplant recipients. Drugs 67, 1463-1481 (2007).
6. R. Yeter, M. Hubler, M. Pasic, R. Hetzer, C. Knosalla, Organ preservation with the organ care system. Applied Cardiopulmonary Pathophysiology 15, 207-212 (2011).
7. G. M. Fahy, B. Wowk, in Cryopreservation and Freeze-Drying Protocols, W. F. Wolkers, H. Oldenhof, Eds. (Springer New York, New York, NY, 2015), pp. 21-82.
8. C.-J. Zhou et al., High survival of mouse oocytes using an optimized vitrification protocol. Scientific Reports 6, 19465 (2016).
9. W. F. Rall, G. M. Fahy, Ice-free cryopreservation of mouse embryos at −196 degrees C. by vitrification. Nature 313, 573-575 (1985).
10. G. M. Fahy et al., Physical and biological aspects of renal vitrification. Organogenesis 5, 167-175 (2009).
11. A. Arav, O. Friedman, Y. Natan, E. Gur, N. Shani, Rat hindlimb cryopreservation and transplantation: a step toward “organ banking”. American Journal of Transplantation 17, 2820-2828 (2017).
12. Z. Wang et al., Cryopreservation and replantation of amputated rat hind limbs. European journal of medical research 19, 28-28 (2014).
13. D. P. Eisenberg, J. C. Bischof, Y. Rabin, Thermomechanical stress in cryopreservation via vitrification with nanoparticle heating as a stress-moderating effect. Journal of Biomechanical Engineering 138, (2015).
14. M. L. Etheridge et al., RF heating of magnetic nanoparticles improves the thawing of cryopreserved biomaterials. Technology 02, 229-242 (2014).
15. M. J. Taylor, Y. C. Song, K. G. M. Brockbank, in Life in the Frozen State. (2004).
16. P. M. Mehl, Nucleation and crystal growth in a vitrification solution tested for organ cryopreservation by vitrification. Cryobiology 30, 509-518 (1993).
17. N. Manuchehrabadi et al., Improved tissue cryopreservation using inductive heating of magnetic nanoparticles. Science Translational Medicine 9, eaah4586 (2017).
18. A. Chiu-Lam, C. Rinaldi, Nanoscale thermal phenomena in the vicinity of magnetic nanoparticles in alternating magnetic fields. Advanced Functional Materials 26, 3933-3941 (2016).
19. J. Wang, G. Zhao, Z. Zhang, X. Xu, X. He, Magnetic induction heating of superparamagnetic nanoparticles during rewarming augments the recovery of hUCM-MSCs cryopreserved by vitrification. Acta Biomaterialia 33, 264-274 (2016).
20. Z. Gao et al., Preparation of Scalable Silica-Coated Iron Oxide Nanoparticles for Nanowarming. Adv Sci (Weinh) 7, 1901624 (2020).
21. H. L. Ring et al., Imaging the distribution of iron oxide nanoparticles in hypothermic perfused tissues. Magn Reson Med 83, 1750-1759 (2020).
22. W. Abdelwahed, G. Degobert, S. Stainmesse, H. Fessi, Freeze-drying of nanoparticles: formulation, process and storage considerations. Advanced Drug Delivery Reviews 58, 1688-1713 (2006).
23. S. Deville, in Freezing Colloids: Observations, Principles, Control, and Use: Applications in Materials Science, Life Science, Earth Science, Food Science, and Engineering, S. Deville, Ed. (Springer International Publishing, Chain, 2017), pp. 91-170.
24. L. C. Wu et al., A review of magnetic particle imaging and perspectives on neuroimaging. American Journal of Neuroradiology, (2019).
25. E. Y. Yu et al., Magnetic particle imaging: a novel in vivo imaging platform for cancer detection. Nano Letters 17, 1648-1654 (2017).
26. R. E. Rosensweig, Ferrohydrodynamics. D. B. o. Physics, Ed., (Dover Publications, 2014), pp. 344.
27. C. Barrera et al., Effect of poly(ethylene oxide)-silane graft molecular weight on the colloidal properties of iron oxide nanoparticles for biomedical applications. Journal of Colloid and Interface Science 377, 40-50 (2012).
28. M. J. Taylor, Y. C. Song, B. S. Kheirabadi, F. G. Lightfoot, K. G. Brockbank, Vitrification fulfills its promise as an approach to reducing freeze-induced injury in a multicellular tissue Advances in Heat and Mass Transfer in Biotechnology 363, 93-102 (1999).
29. K. G. M. Brockbank, Z. Chen, E. D. Greene, L. H. Campbell, in Cryopreservation and Freeze-Drying Protocols, W. F. Wolkers, H. Oldenhof, Eds. (Springer New York, New York, NY, 2015), pp. 399-421.
30. Z. W. Tay et al., Magnetic Particle Imaging-Guided Heating in Vivo Using Gradient Fields for Arbitrary Localization of Magnetic Hyperthermia Therapy. ACS Nano 12, 3699-3713 (2018).
31. Y. C. Song, B. S. Khirabadi, F. Lightfoot, K. G. M. Brockbank, M. J. Taylor, Vitreous cryopreservation maintains the function of vascular grafts. Nature Biotechnology 18, 296-299 (2000).
32. F. Merida et al., Optimization of synthesis and peptization steps to obtain iron oxide nanoparticles with high energy dissipation rates. Journal of magnetism and magnetic materials 394, 361-371 (2015).
33. B. S. Lele, M. G. Kulkarni, Single step room temperature oxidation of poly(ethylene glycol) to poly(oxyethylene)-dicarboxylic acid. Journal of Applied Polymer Science 70, 883-890 (1998).
34. M. J. Taylor, B. P. Weegman, S. C. Baicu, S. E. Giwa, New Approaches to Cryopreservation of Cells, Tissues, and Organs. Transfusion medicine and hemotherapy: offizielles Organ der Deutschen Gesellschaft fur Transfusionsmedizin und Immunhamatologie 46, 197-215 (2019).

### Example 2

Superparamagnetic iron oxide nanoparticle (SPION) tracers possessing long blood circulation time and tailored for magnetic particle imaging (MPI) performance are crucial for the development of this emerging molecular imaging modality. Here, single-core SPION MPI tracers coated with covalently bonded polyethyelene glycol (PEG) brushes were obtained using a semi-batch thermal decomposition synthesis with controlled addition of molecular oxygen, followed by an optimized PEG-silane ligand exchange procedure.

The physical and magnetic properties, MPI performance, and blood circulation time of these newly synthesized tracers were compared to those of two commercially available SPIONs that were not tailored for MPI but are used for MPI: ferucarbotran and PEG-coated Synomag®-D. The new tailored tracer has MPI sensitivity that is ˜3-times better than the commercial tracer ferucarbotran and much longer circulation half-life than both commercial tracers (t_{1/2}=6.99 h for the new tracer, vs t_{1/2}=0.59 h for ferucarbotran, and t_{1/2}=0.62 h for PEG-coated Synomag®-D).

### INTRODUCTION

Magnetic particle imaging (MPI) has attracted tremendous interest as a molecular imaging modality since it was first reported in 2005 [1]. In MPI, a uniform alternating magnetic field (AMF) is applied to a field of view while opposing magnets are used to create a quasistatic selection field gradient with a small field free region (FFR). Superparamagnetic iron oxide nanoparticles (SPIONs) located in the FFR respond to the applied AMF and generate a signal that can be recorded using pickup coils, while SPIONs outside the FFR are unable to respond to the AMF due to saturation caused by the selection field gradient [1, 2]. The signal generated by SPIONs at the FFR is proportional to its mass and a quantitative 3D distribution of the SPIONs can be determined by moving the FFR to cover a field of view (FOV) of interest. Signal generation in MPI relies on the nonlinear superparamagnetic response of the SPIONs resulting in negligible signal from tissue, bones, and air gaps. Furthermore, there is negligible tissue attenuation of the magnetic fields used for MPI and of the signal generated by the SPIONs, resulting in images with negligible tissue depth limitations [1, 2]. This combination of features makes MPI an ideal approach for unambiguous and sensitive non-invasive quantification of SPION biodistribution. In addition, because SPIONs can be used to label cells and other biomaterials, MPI has tremendous potential for applications such as cell tracking [3], nanoparticle drug [7], and blood pool imaging [8, 9].

The sensitivity and resolution achievable in MPI arise due to a combination of hardware, software, and the magnetic properties of the SPION tracer [10]. The introduction of commercial pre-clinical MPI scanners has supported a wide range of studies seeking to apply MPI in novel biomedical settings and there is a tremendous need for SPION tracers with suitable MPI properties. Importantly, the physics of signal generation in MPI are distinct from that responsible for SPION contrast enhancement in magnetic resonance imaging (MRI). In MPI signal arises directly and solely from the non-linear magnetization response of SPIONs to the excitation field. In MRI, SPION contrast enhancement arises due to changes in proton relaxivity when they are in close proximity to SPIONs. Importantly, in MRI the SPIONs do not respond to the pulsed field because they are in a saturated state. As such, SPION tracers developed for MRI are not necessarily ideal for MPI. Furthermore, in addition to the magnetic properties of the SPIONs, surface modification and formulation must be tailored for specific applications. For example, there are several applications of MPI that would benefit from SPION tracers with long circulation lifetimes, including blood pool imaging [8, 9], functional MPI [11], cancer imaging [12], evaluate traumatic brain injury [7], and in vivo gut bleed detection [8]. These considerations suggest a need for developing SPIONs with physicochemical and magnetic properties that are tailored for specific MPI applications.

Several commercially available SPIONs have been studied as MPI tracers. Ferucarbotran (an off-brand version of Resovist®) is a commercially available SPION contrast agent developed specifically for MRI that is commonly used for MPI studies [13, 14]. However, it has been suggested that only 3% of the total iron mass from Resovist® contributes to the MPI signal due to particle-particle interaction within carboxydextran coated core [1]. Another commercially available tracer of potential use for MPI is Synomag®-D, which consists of multi-core SPIONs. Performance of Synomag®-D in MPI has been evaluated using a magnetic particle spectrometer (MPS) and the results suggested better performance compared to Resovist® [15]. Synomag®-D has been used to image flow in phantoms [16] and to label erythrocytes and cancer cells [17, 18]. However, studies evaluating performance of Synomag®-D in vivo are lacking. SPION tracers have also been developed specifically for use in MPI. An example of a tracer developed specifically for MPI is LS-008, from LodeSpin Labs, LLC, which combined high sensitivity and resolution with a circulation half-life of ˜105 min in mice [10, 19, 20]. However, LS-008 is no longer available. Furthermore, while these tracers have been widely tested using academic prototype MPI scanners and with the Bruker pre-clinical MPI scanner, their performance has not been evaluated in the newer Magnetic Insight, Inc., MOMENTUM™ pre-clinical scanner. Because SPION performance varies with the configuration of the magnetic field, the magnitude of the field gradient in the FFR, and the amplitude and frequency of the AMF used to excite the SPIONs, MPI performance of a given tracer is expected to vary from one type of scanner to another. The growing adoption of the MOMENTUM™ MPI scanner suggests that comparative performance studies of MPI tracers using this scanner would be of value to the community.

In this example the synthesis, surface modification, and MPI performance of a new tracer (denoted as RL-1) tailored for MPI which possesses a long blood circulation half-life (˜7 hour), suitable for blood pool imaging applications and other applications where long blood circulation time is desirable is provided. The MPI performance and pharmacokinetics of the new RL-1 tracer are compared to those of the commercially available tracers ferucarbotran and Synomag®-D coated with polyethylene glycol. The SPIONs in this tracer were synthesized by thermal decomposition with addition of molecular oxygen [21], and subsequently coated with a covalently grafted layer of polyethylene glycol (PEG). Physical, magnetic, and hydrodynamic properties of the RL-1 tracer and the two commercial tracers were evaluated. The MPI performance (resolution, signal per unit Fe mass, and limit of detection) of all tracers and their pharmacokinetics in mice were evaluated using the MOMENTUM™ MPI scanner.

### METHODS AND MATERIAL

### Materials

Iron (III) acetylacetonate (>98% pure) and 3-aminopropyl triethoxysilane (APS, >98.0%) were purchased from TCI America (Portland, OR). Oleic acid (90% technical grade), docosane (90% pure), 1-octadecene (90% technical grade), polyethylene glycol monomethyl ether (mPEG, 5 kDa), sulfuric acid (99.999%), isopropyl alcohol (70%), tetra(ethylene glycol) dimethacrylate (TEGDMA, 90%), and 2,2′-Azobis(2-methylpropionitrile) (98%), potassium nitrate (>99%, ACS reagent), glycerol (>99%), were purchased from Sigma-Aldrich (St. Louis, MO). Toluene (>99.5%, ACS reagent), ethanol (200 proof), chromium trioxide (certified ACS), acetone (certified ACS), diethyl ether (certified ACS), hydrochloric acid (37% w/v), 1-ethyl-3-(3-dimethylaminopropyl) carbodiimide (EDC), activated charcoal (12-40 mesh), acetone (certified ACS), diethyl ether (ACS chemical, BHT stabilized), dichloromethane (99.6%, ACS reagent), nitric acid (Certified ACS Plus), potassium hydroxide (85%, ACS reagent), and CBQCA protein quantitation kit were purchased from Thermo Fisher Scientific (Waltham, MA). N-hydroxysulfosuccinimide (sulfo-NHS) was purchased from ProteoChem™ (Hurricane, UT). Magnetic columns were purchased from Miltenyi Biotec (Germany). Ferucarbotran was purchased from Meito Sangyo Co., LTD (Japan). Synomag®-D, coated with PEG 25000-OMe, 50 nm, was purchased from micromod Partiketechnologie GmbH (Germany). Copper TEM grid (carbon film only, 200 mesh) was purchased from TED PELLA, INC (Redding, CA).

### Particle Synthesis

### Synthesis of Iron (III) Oleate:

A stoichiometrically defined iron oleate was prepared according to published work with modifications [21]. Iron acetylacetonate (22.38 g, 63.36 mmol) and oleic acid (89.48 g, 316.80 mmol) were added to a 500 ml three-neck reactor. The flask was equipped with an overhead stirrer in the middle neck, a septum with a thermocouple and a stainless steel (SS) needle through the left neck. Argon (100 sccm) was supplied continuously through the SS needle during synthesis, using a mass flow controller. A condenser connecting to a chiller was attached in the right neck. A molten metal bath and temperature controller was used as the heating source. The molten metal was heated to 110° C. before pushing the reaction vessel into the molten metal bath. The reaction mixture was then heated to 310° C. under stirring at 350 rpm. Thirty minutes after the reaction mixture reached 300° C., the reaction was stopped to obtain a dark brown waxy liquid. The resulting iron oleate was purged using argon and stored until use for nanoparticle synthesis.

### Synthesis of Magnetic Iron Oxide Nanoparticles:

Docosane (10.1 g, 32.23 mmol) and oleic acid (6.23 g, 22.06 mmol) were added to a 100 ml three-neck reactor. Separately, iron oleate was mixed with 1-octadecene (27.12 g, 107.40 mmol) to prepare a precursor with 0.22 M Fe. The flask was equipped with an overhead stirrer in the middle neck and a septum with a SS needle through the left neck. A molten metal bath and temperature controller was used as the heating source. The molten metal was heated to 110° C. before pushing the reaction vessel into the molten metal bath. The mixture was heated to 350° C. in 30 mins before the controlled addition of iron oleate precursor (1.98 to 2.64 mmol/h) using a syringe pump. Nitrogen (100 secm) was supplied continuously through the SS needle, controlled using a mass flow controller until the reaction mixture reached 350° C. Then, 140 scm of 1% oxygen in argon mixture was introduced into the reactor headspace through the SS needle, controlled using a mass flow controller. Uniform mixing at 350 rpm was maintained throughout the reaction. The precursor drip was stopped after 4 to 5 hours and the reactor was removed from the molten metal bath. Toluene and ethanol in a 2:3 volume ratio were used to precipitate nanoparticles from the crude synthesis product. Purified oleic acid coated particles were suspended in toluene and stored at 4° C.

### Particle Coating

### PEG-Silane Synthesis:

A polyethylene glycol-silane conjugate (PEG-silane) was synthesized via a two-step procedure. First, mPEG (5 kDa) was converted to mPEG acetic acid (mPEG-COOH) using a strong oxidizing agent [22]. Briefly, 50 g of mPEG was dissolved in 400 mL of acetone. Jones reagent (prepared using 70 g of chromium trioxide in 500 mL of deionized water and 71 mL of sulfuric acid) was used to oxidize mPEG. Once the mPEG was dissolved in acetone, 16.1 mL of Jones reagent was added and allowed to react for 24 hours. Approximately, 5 mL of isopropyl alcohol was added to stop the reaction and 5 g of activated charcoal was added to remove impurities. The chromium salts and activated charcoal were removed using vacuum filtration. The acetone solution containing the oxidized mPEG was concentrated using a rotary evaporator. The concentrated mixture of mPEG-COOH was re-dissolved in 50 mL of 1 M HCl. The polymer was then extracted to the organic phase by liquid-liquid extraction using 150 mL dichloromethane. The extraction allows for removal of chromium trioxide since it is insoluble in dichloromethane. The solution was concentrated by rotary evaporation. The concentrated mPEG-COOH was precipitated using cold diethyl ether. The mPEG-COOH was then dried in a vacuum oven at room temperature. Proton nuclear magnetic resonance (NMR) spectroscopy was used to ascertain there was full conversion of mPEG to mPEG-COOH. Second, mPEG-COOH was amidated by reaction with APS to obtain PEG-silane. Briefly, mPEG-COOH was weighed and melted in an oil bath set to 60° C. Then, APS was added to the melted PEG at a 1:1 molar ratio. The mixture was allowed to react for 2 hours at 120° C. and 500 mbar. The PEGsilane was then cooled to room temperature and collected. The resulting PEG-silane was analyzed through gel permeation chromatography (GPC).

### Ligand Exchange

SPIONs were coated with PEG-silane using ligand exchange, replacing the oleic acid on the surface of the SPIONs with PEG-silane, following procedures similar to Zhu et al [23]. Briefly, 0.7 g of PEG-silane was dissolved in 4 mL of dry toluene. Once the PEG-silane was dissolved, 2 mL of SPIONs at 2.5 mg Fe₃O₄ per mL and 28 μL of APS were added and mixed. The solution was capped and allowed to react overnight, approximately 16 hours, in a heating block set at 100° C. The next day, the PEG-silane coated SPIONs were precipitated out of solution using cold diethyl ether. The sample was centrifuged and supernatant discarded. The SPIONs were resuspended in acetone and precipitated again with cold diethyl ether twice. The precipitate was then dried in a vacuum oven at room temperature overnight. The following day, PEG-silane coated SPIONs were resuspended in water and dialyzed to remove excess PEG-silane. For further purification, particles were purified using magnetic columns. The resulting nanoparticles were backfilled with additional PEG-COGH using EDC-NHS chemistry[24]. The number of remaining primary amines on the particles was quantified using the CBQCA protein quantification kit, following the manufacturer's protocol. Once the number of amines were determined, a ratio of 1:2 amine to carboxylic acid was used. The mPEG-COOH was suspended in water and pH adjusted to 5.0. EDC was added at a 1:2 carboxylic acid:EDC ratio and allowed to react for 15 minutes. Then, sulfo-NHS was added at a 1:1 ratio of EDC to sulfo-NHS. The pH of the solution was slowly adjusted to 7.0 and reacted for 15 minutes. Last, the nanoparticle solution was added and the pH adjusted to 9.0. The mixture reacted overnight and was purified using a magnetic column. Finally, the nanoparticles were sterilized using a 0.22 μm PES syringe filter.

### Physical and Magnetic Characterization

### Transmission Electron Microscopy

Images of iron oxide particles sampled on 200-mesh copper grids with carbon film were acquired using a FEI Talos F200i S/TEM. Physical diameters (Dp) were obtained by analyzing the images using Fiji [25]. Reported size distribution statistics and histograms are based on at least 2000 particles for RL-1 nanoparticles, or at least 400 particles for ferucarbotran and Synomag©-D.

The number median diameter (D_{pg}) and geometric standard deviation (ln ag) of the particle size distribution were obtained by fitting the size distribution histograms to the lognormal distribution (n_{N}(Dₚ)) using [21]

$${n}_{N} ( {D}_{p} ) = \frac{1}{\sqrt{2 π} {D}_{p} ln {σ}_{g}} exp ( - \frac{{ln}^{2} {D}_{p} / {D}_{p g v}}{2 {ln}^{2} {σ}_{g}} ) \qquad ( 1 )$$

D_{pg} was converted to a volume median diameter (D_{pgv}) using [21]

D_{pgv}=exp[ln D_{pg}+3 ln²σ_{g}] (2)
The arithmetic volume weighted mean diameter (Dₚᵥ) and standard deviation (σ) were calculated using [21]

$${D}_{p v} = exp ( ln {D}_{p g v} + \frac{{ln}^{2} {σ}_{g}}{2} ) \qquad ( 3 )$$

σ=Dₚᵥ\sqrt{exp(ln²σ_{g}−1)} (4)

### Dynamic Light Scattering and Zeta Potential:

A Brookhaven Instruments 90Plus/BI-MAS dynamic light scattering and zeta potential measurement instrument, operating at a scattering angle of 900 at room temperature, was used to determine the hydrodynamic size and zeta potential of the SPIONs. For hydrodynamic diameter measurements, particles were suspended at 1 mg/mL in deionized water. The zeta potential of the particles was measured in a 1 mM KNO₃ solution at pH 7, adjusted with nitric acid and potassium hydroxide.

### Magnetometry:

Magnetic characterization was performed with the particles suspended in water at 300 K to obtain magnetization data for the purpose of bimodal magnetic diameter fitting. Particle concentration was ˜1 mg Fe/ml, according to the 1,10-phenanthroline spectrophotometric assay [26].

Nanoparticles were also embedded in a TEGDMA matrix using a technique described previously in order to perform more detailed magnetic characterization [27]. These characterizations included magnetization versus magnetic field (MH) curves taken at 295, 305 and 315 K, plotted as a function of the ratio of magnetic field to absolute temperature, to verify superparamagnetic behavior. Additionally, the zero field cooled/field cooled measurements were used to obtain blocking temperatures for the estimation of magnetic anisotropy constant. The MH curves obtained at 315 K were used to obtain the monomodal magnetic diameter estimate used in determination of the anisotropy constant as well. To prepare samples embedded in TEGDMA, a concentrated nanoparticle suspension in water was mixed with TEGDMA monomer at a particle concentration of 0.1 wt %. Then, the initiator 2,2′-Azobis(2-methylpropionitrile) was added at a concentration of 0.05 wt %, and crosslinking was performed by heating the mixture at 70° C. for 6 h.

DC equilibrium magnetization curves of the particles in water and in the hard polymer matrices were obtained using a magnetic property measurement system (MPMS-3) superconducting quantum interference device (SQUID) magnetometer (Quantum Design, Inc. CA, USA). Samples were mounted in the instrument using PTFE sample holders for suspensions and plastic straws for polymer matrices.

Magnetic Diameter fitting for liquid samples: The volume-weighted median magnetic diameters (Dₘᵥ) and geometric deviation (ln σ_{g}) of the iron oxide nanoparticles suspended in water at 300 K were obtained by fitting the measured magnetization data M(H) to the Langevin function L(α) for superparamagnetism, weighted using a bimodal lognormal size distribution. The single population lognormal weighting for the Langevin function suggested by Chantrell et al [28], was modified to a bimodal distribution by considering that the particle magnetic diameter population was well-represented by the sum of two single modal lognormal distributions. In the equations below, Mₛ is the saturation magnetization of the sample, ϕ₁ is the mass fraction of the first diameter distribution, nᵥ₁(Dₘ) and nᵥ₂(Dₘ) are the lognormal distribution functions, a is the Langevin parameter, Wis the permeability of free space, M_{d} is the domain magnetization (446,000 A/m for bulk magnetite [29]), k_{B} is Boltzmann's constant, and T is the measurement temperature. D_{mv,1} and D_{mv,2} are the volume weighted median diameters of the two magnetic diameter distributions, and In σ_{g,1} and In G_{g,2} are the geometric deviations. The fit was performed in MATLAB® (MathWorks, MA, USA) using a non-linear regression model. The arithmetic volume weighted mean diameter (Dₘᵥ) and standard deviation (σ) were calculated using Equation (3) and (4).

$$M ( H ) = {M}_{S} {\int }_{0}^{∞} [ {ϕ}_{1} {n}_{v 1} ( {D}_{m} ) + ( 1 - {ϕ}_{1} ) {n}_{v 2} ( {D}_{m} ) ] L ( α ) d {D}_{m} \qquad ( 5 )$$

$${n}_{v , 1} ( {D}_{m} ) = \frac{1}{\sqrt{2 π} {D}_{m} ln {σ}_{g , 1}} exp [ - \frac{{ln}^{2} {D}_{m} / {D}_{mv , 1}}{2 {ln}^{2} {σ}_{g , 1}} ] \qquad ( 6 )$$

$${n}_{v , 2} ( {D}_{m} ) = \frac{1}{\sqrt{2 π} {D}_{m} ln {σ}_{g , 2}} exp [ - \frac{{ln}^{2} {D}_{m} / {D}_{m v , 2}}{2 {ln}^{2} {σ}_{g , 2}} ] \qquad ( 7 )$$

$$L ( α ) = coth ( α - \frac{1}{α} ) \qquad ( 8 )$$

$$α = \frac{π {μ}_{0} {M}_{d} {D}_{m}^{3} H}{6 {k}_{B} T} \qquad ( 9 )$$

### Estimation of Effective Anisotropy Constant:

The volume-weighted median magnetic diameters (Dₘᵥ) and geometric deviation (ln σ_{g}) of the iron oxide nanoparticles embedded in solid matrix were obtained by fitting the magnetization data to the Langevin function L(α) for superparamagnetism, weighted using a monomodal lognormal size distribution, as suggested by Chantrell et al [28]. Note that bimodal size distributions were used in the case of liquid samples because they match the measured magnetization data with more fidelity. In contrast, here we chose a monomodal distribution because the model used to estimate the effective anisotropy constant does not account for multiple size distributions. In equations (10) and (11) below, nᵥ(Dₘ) is the lognormal distribution function, Mₛ is the saturation magnetization of the sample, and a is the Langevin parameter, defined in equation (9).

$$M ( H ) = {M}_{S} {\int }_{0}^{∞} {n}_{v} ( {D}_{m} ) L ( α ) d {D}_{m} \qquad ( 10 )$$

$${n}_{v} ( {D}_{m} ) = \frac{1}{\sqrt{2 π} {D}_{m} ln {σ}_{g}} exp [ - \frac{{ln}^{2} {D}_{m} / {D}_{m v}}{2 {ln}^{2} {σ}_{g}} ] \qquad ( 11 )$$

The fit was performed in MATLAB using a non-linear regression model. Zero field cooled and field cooled (ZFC-FC) magnetization measurements were made to obtain the blocking temperature (T_{B}) for each nanoparticle. Samples embedded in polymer matrices were prepared as described above. At the start of the measurements, samples were first heated to 400 K at zero field, and then cooled to 4 K at zero field. A field of 10 Oe was applied and the magnetization measured as the temperature was swept at 2 K/min from 4 K to 400 K in the ZFC portion of the curve. Then, for the FC portion of the curve, the sample was cooled to 4 K at 2 K/min while the magnetization of the sample was measured. The value of the blocking temperature T_{B} was estimated by applying a simple parabolic fit to the portion of the ZFC curve where the peak in measured magnetization occurred. Equation (12) below was then used to calculate the effective anisotropy constant using the Néel model, accounting for the dispersity in magnetic diameters [27]. Here, Kₘ is the effective magnetic anisotropy constant of the particles, k_{B} is the Boltzmann's constant, T_{B} is the blocking temperature, Dₘᵥ is the volume weighted median magnetic diameter from the monomodal fit performed above, τ_{obs} is the observation time, τ₀ is the attempt frequency (assumed widely to be 10⁻⁹ s), ln σ_{g} is the geometric deviation of the magnetic diameter distribution obtained above, and Tᵣₐₜₑ is the temperature sweep rate of our ZFC/FC measurements, equal to 2 K/min in all measurements performed.

$${K}_{m} = \frac{6 {k}_{B} ( {T}_{B} )}{π {D}_{m v}^{3}} ln ( \frac{{τ}_{o b s}}{{τ}_{0}} ) \frac{1}{exp ( \frac{9}{2} {ln}^{2} {σ}_{g} )} \qquad ( 12 )$$

### Dynamic Magnetic Susceptibility:

The dynamic magnetic susceptibility of all tracers in liquids (200 μl of total volume) of different viscosities were measured using a DynoMag AC susceptometer (Rise Research Institutes, Sweden) in a small amplitude oscillating magnetic field at a constant temperature and as a function of the frequency of the oscillating magnetic field. Measurements were made in deionized water and in a 65% w/w glycerol in water solution (viscosity=0.0125 Pa s) in order to evaluate the mechanism of magnetic relaxation of the particles.

### MPI Performance

### Sample Holder Design:

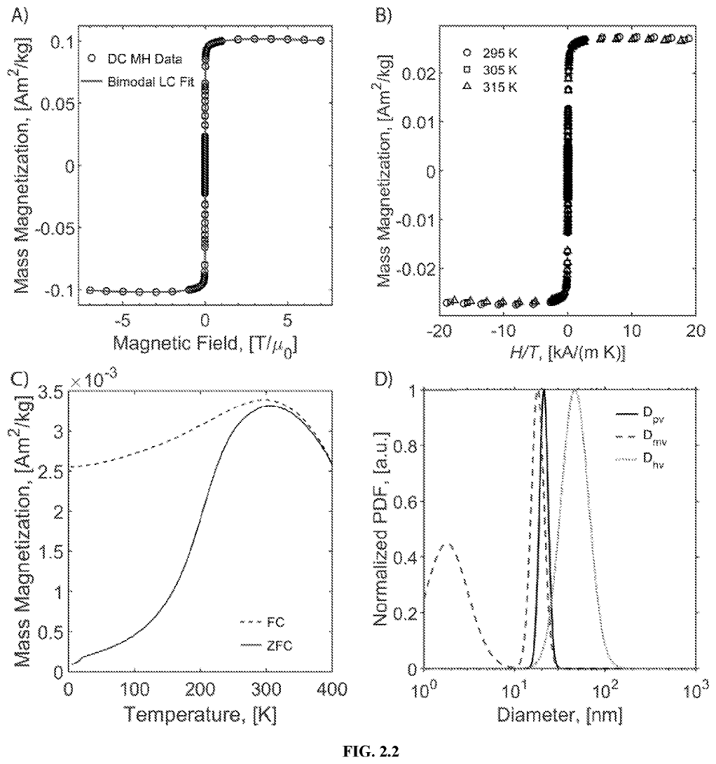

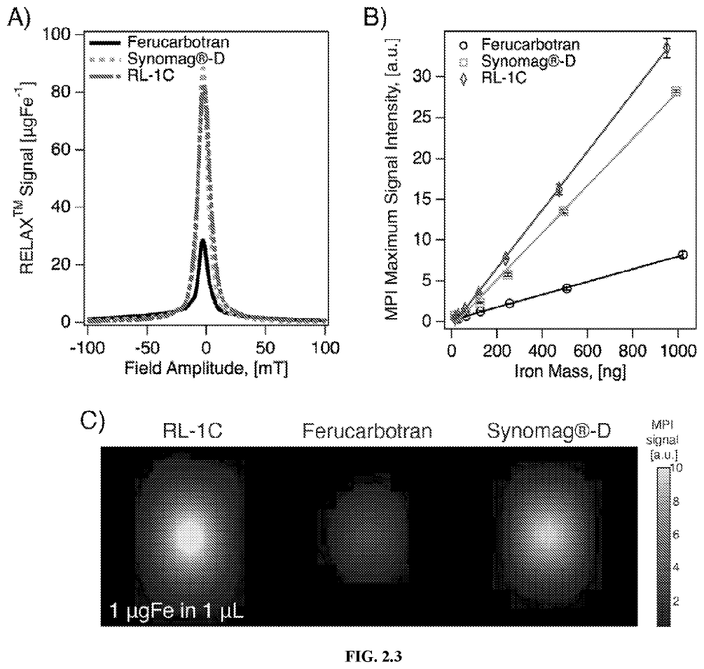

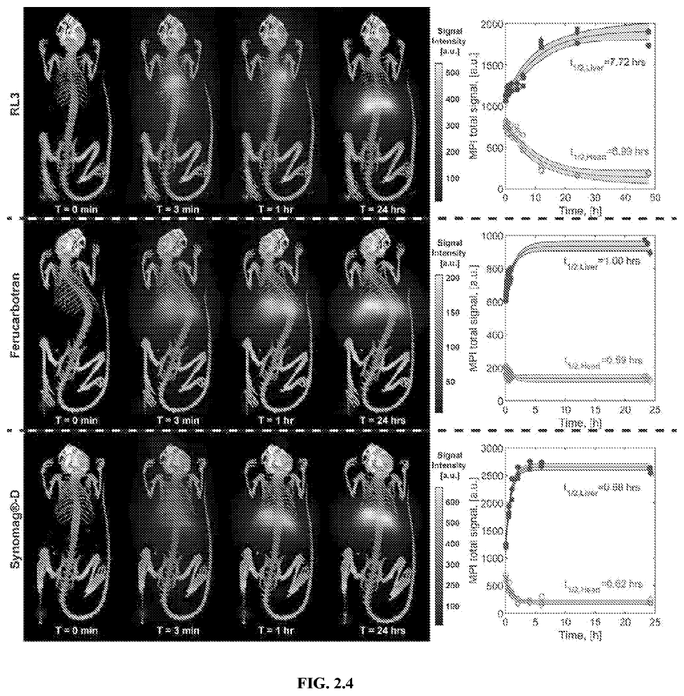

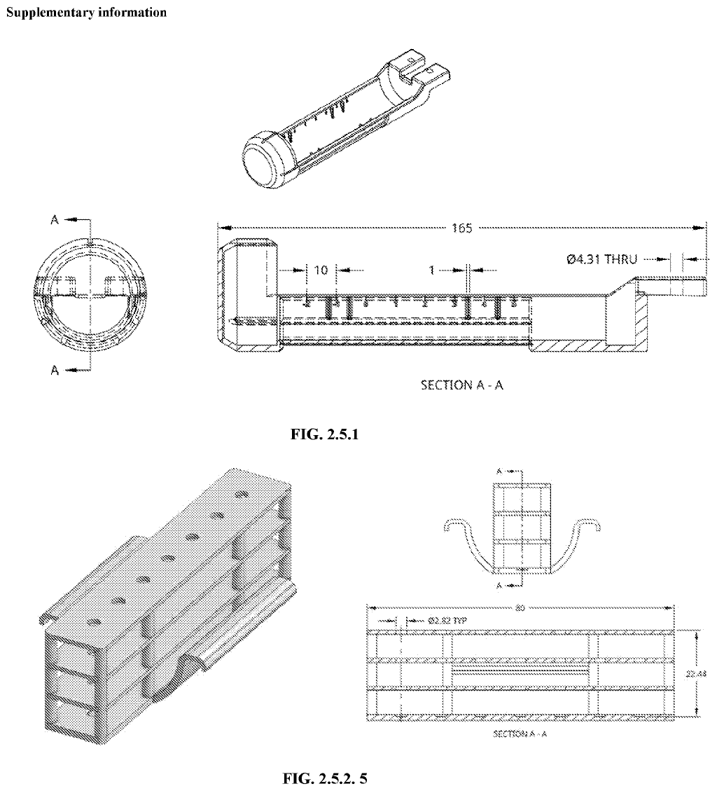

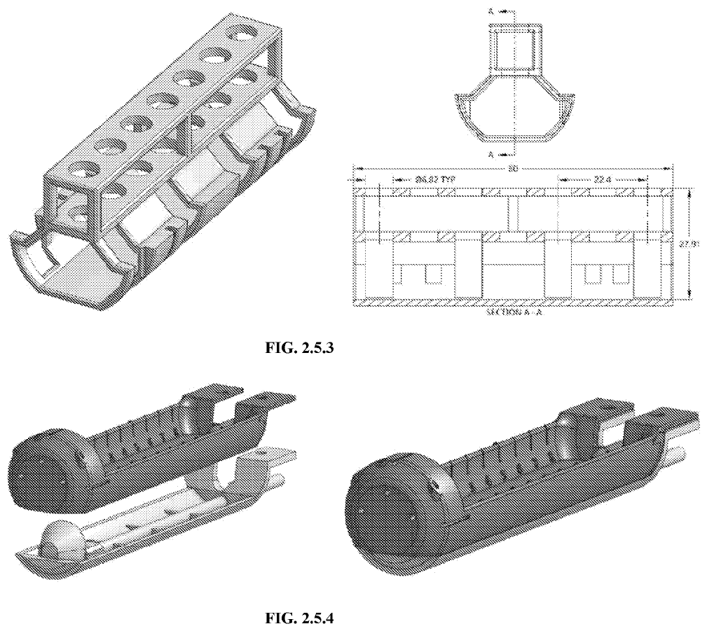

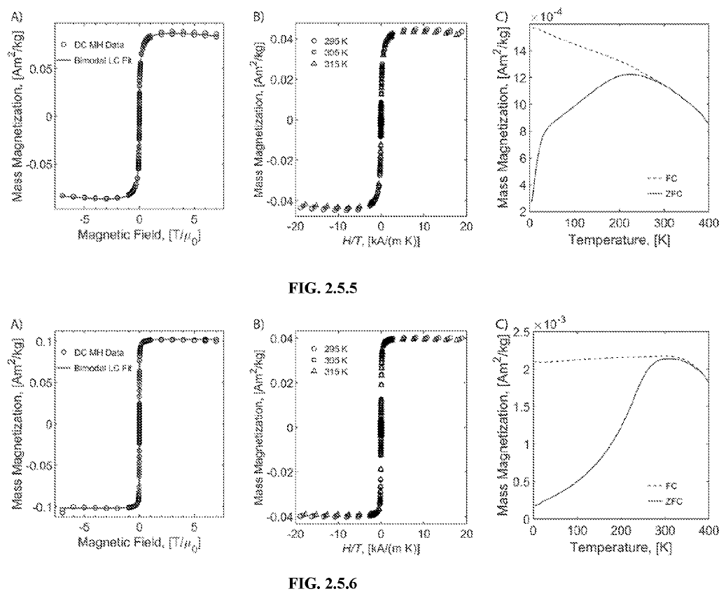

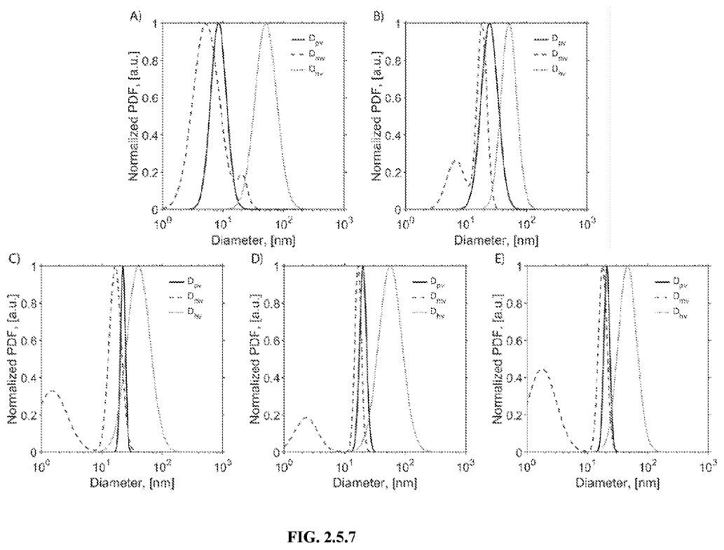

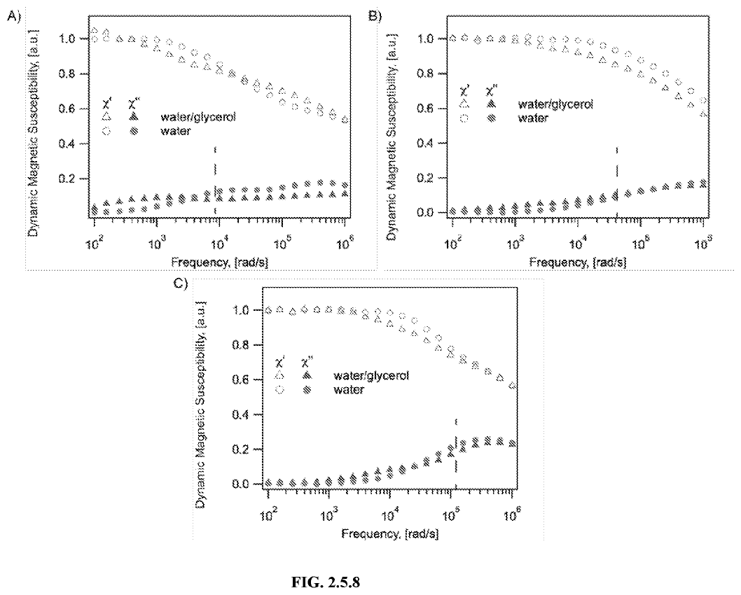

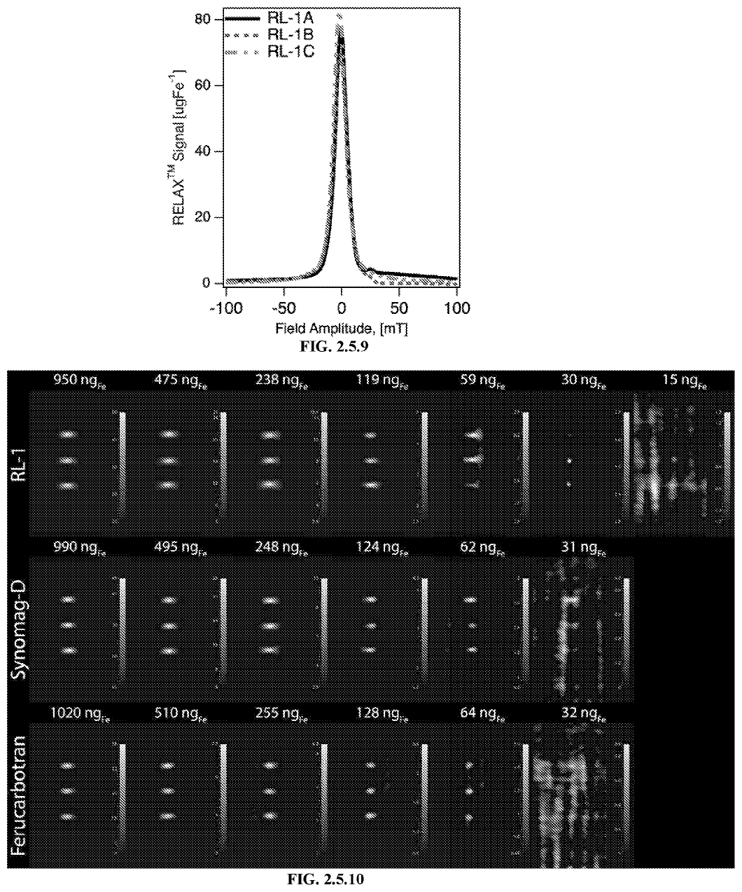

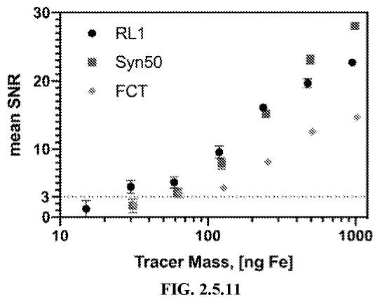

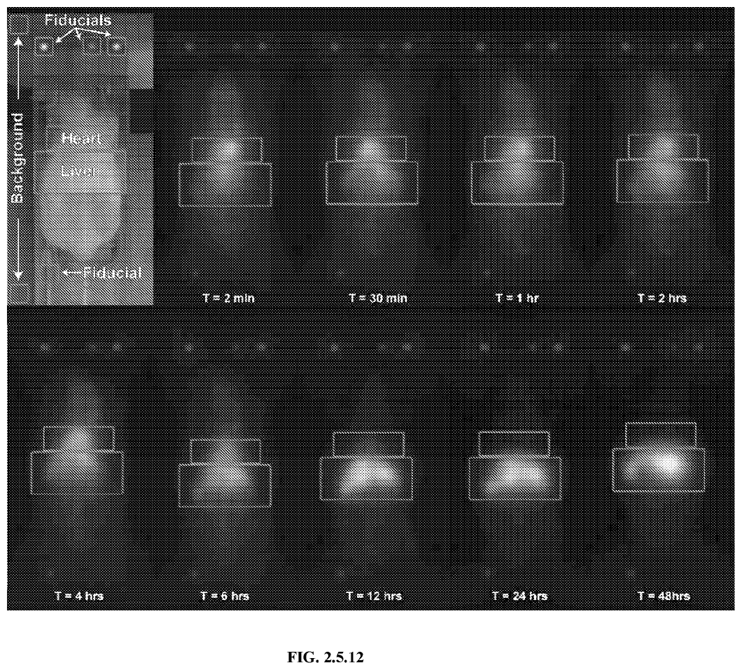

To image each sample in the MPI scanner, novel sample holders were designed using the online, three-dimensional (3D) computer aided design program Onshape (Onshape, MA, USA) and 3D printed with the Form 3 stereolithography printer (Formlabs, MA, USA). Each sample holder was designed as a removable part that fits inside a customized 3D printed bed which was attached to the MPI scanner arm. This custom bed design is shown in FIG. 2.5.1. The parts used in MPI scans were printed using Clear V4 resin (Formlabs, MA, USA) with layer sizes ranging from 25-100 μm.

The design of the capillary tube holder used for limit of detection (LoD) testing is shown in FIG. 2.5.2. This design holds up to 7 tubes oriented vertically and arranged in a linear pattern along the z-scan direction in the FOV. The tube bores are 2.82 mm in diameter with a center-to-center spacing of 10.35 mm.

Relax scan measurements performed on samples in 0.2 mL microcentrifuge tubes utilized sample holders which held the tubes vertically. Similar to the capillary tube holders, these microcentrifuge tube holders each hold up to 7 tubes centered within the 80 mm sample holder length. Each microcentrifuge tube rests in a 6.82 mm diameter bore, and the bores have a center-to-center spacing of 11.20 mm. The vertical configuration of this model is shown in FIG. 2.5.3.

### Mpi Measurements:

To determine the LoD for each SPION, we first determined the iron concentration using the 1,10-phenanthroline colorimetric assay [26]. Dilution series were prepared using a dilution factor of two, with concentrations ranging from 1000 μg_{Fe}/ml to 15 μg_{Fe}/ml. All samples consisted of 1 μL of solution (containing 1 μg_{Fe} to 15 ng_{Fe}) in a capillary tube (1/32″ ID) placed parallel to the y-axis in the field of view (FOV). Each concentration was acquired in triplicate by placing three capillary tubes featuring the same iron mass in the FOV (6×12 cm). MPI scans were acquired with the MOMENTUM™ scanner (Magnetic Insight, CA, USA) using high-sensitivity (3 T/m) multichannel scan mode (x- and z-channel scans).

Images were analyzed using MATLAB® (MathWorks, MA, USA) in-house algorithms in which the region of interest (ROI) was selected and maximum signal intensity was obtained. Images were also analyzed using 3D Slicer [30, 31]. The LoD was evaluated several ways. First, an LoD was calculated by analyzing the background signal from empty scans, the limit of blank (LoB), and the maximum intensity signal of samples at low concentrations, using the equations [32]:

LoB=mean_{blank}+1.645(SD_{blank}) (13)

LoD=LoB+1.645(SD_{low concentration sample}) (14)

The LoD was also evaluated based on calculation of mean signal to noise ratio (mSNR), calculated as the ratio of the mean signal intensity in the ROI to the standard deviation of the background region for each scan. Finally, the LoD was evaluated by inspection of the individual scans to confirm that the signal corresponded to the dilution samples and not to background signal fluctuations.

For relax scan measurements, 10 μL of each particle (RL-1, Ferucarbotran, and Synomag®-D) were placed in a 0.2 mL microcentrifuge tube, and the sample was centered in the FOV. Then, the x-space point spread function (PSF) was measured using the RELAX™ modality in the MOMENTUM™ scanner. The PSF was normalized by the iron mass to facilitate comparison of different particles. The signal intensity was reported by normalizing the system reported amplitude using iron mass and the FWHM is the system reported value.

### Tracer Blood Circulation Time Evaluated Via MPI

### Animal Models:

All animal procedures were conducted according to the protocols approved by the Institutional Animal Care and Use Committee at the University of Florida. Female Balb/c (6 weeks old) were obtained from Envigo (Indianapolis, IN). All animals were acclimatized for at least one week prior to experimentation.

### Tracer Administration and Imaging:

Female Balb/c mice 8-11 weeks old were used for all the experiments. SPIONs were dispersed in sterile PBS 1× and filtered with 0.22 μm PES filters prior to intravenous administration. Mice were injected with 200 μL of 1 mg_{Fe}/mL as a bolus injection in the lateral tail vein using a 28 G insulin syringe (n=3). Mice were anesthetized immediately with 4-5% isoflurane in an induction chamber and then maintained at 1-2% for the duration of the imaging period. Imaging was performed using MOMENTUM™ scanner (Magnetic Insight, CA, USA) for quantitative imaging. Animals were placed in a 3D printed animal bed (FIG. 2.5.4) designed to be compatible with the MOMENTUM™ MPI scanner and the Perkin Elmer IVIS SpectrumCT imager used to acquire anatomical CT images. Imaging was done in high sensitivity/high resolution (5.7 T/m) scan mode and a 12 cm×6 cm×6 cm FOV. Scans were performed at the following time intervals for RL1-C (0, 0.5, 1, 2, 4, 6, 12, 24, and 48 h), ferucarbotran (every 2 min for the first 1 h and at 24 h), and Synomag®-D (0, 0.5, 1, 2, 4, 6, and 24 h). Acquisition times were 3 min for 2D scans and 42 minutes for 3D scans.

### Quantitative Analysis of MPI:

SPIONs distribute rapidly in the circulatory system once injected. Two regions of interest (ROI) were selected: one for the heart to represent particles in circulation and another for the liver, as the compartment in which most SPIONs will deposit. For each image, a threshold value was used to eliminate inherent background signal from the bed and instrument. The threshold value was determined using the maximum intensity pixel of an empty bed scan. After this threshold was applied to all images, an ROI was drawn over the heart and liver/spleen region. The size of the ROIs was equal for all the images. The reported MPI signal was the total signal in the ROI. To determine tracer half-life, MPI signal for each ROI was fitted to a simple one compartment model using a nonlinear least-squares method.

$${S}_{heart} = {S}_{∞ , heart} + ( {S}_{0 , heart} - {S}_{∞ , heart} ) exp ( - \frac{ln ( 2 ) t}{{t}_{1 / 2 , heart}} ) \qquad ( 15 )$$

$${S}_{liver} = {S}_{∞ , liver} + ( {S}_{0 , liver} - {S}_{∞ , liver} ) exp ( - \frac{ln ( 2 ) t}{{t}_{1 / 2 , liver}} ) \qquad ( 16 )$$

where Sₕₑₐᵣₜ is the signal in the heart ROI, Sₗᵢᵥₑᵣ is the signal in the liver ROI, t is time, S_{∞,heart} and S_{∞,liver} are the long-time signals in the heart and liver ROI, accounting for residual background signal, S_{0,heart} and S_{0,liver} are the initial signals in the heart and liver ROI, and t_{1/2,heart} and t_{1/2,liver} are the characteristic half-lives for particle clearance from the blood circulation and for particle accumulation in the liver, respectively. The 95% confidence interval was evaluated for the estimated half-lives. All image processing and analysis was performed using MATLAB®(MathWorks, MA, USA).

### Image Registration:

Anatomic CT (IVIS SpectrumCT, Perkin Elmer, MA, USA) reference images were acquired on anesthetized animals in standard-one mouse mode with voxel size of 150 μm and resolution of 425 μm (20 ms exposure time, 440 AI X-Ray filter). Image registration was established using fiducials that contained a mixture of SPIONs (MPI tracer) and Omnipaque™ (CT tracer) as markers to align the MPI data with the CT maximum projection image. MPI-CT 2D images were registered using MATLAB®(MathWorks, MA, USA) while 3D registration and visualization was performed using 3D Slicer the landmark registration module and using maximum intensity projection for volume rendering [30, 31].

### Results

### Comparison of Tracer Physical and Magnetic Properties

Iron oxide nanoparticles were synthesized through the thermal decomposition of iron oleate in the presence of molecular oxygen. Three batches of particles (RL-1A, RL-1B, RL-1C) were obtained to illustrate reproducibility over physical and magnetic properties. Then their properties were compared to two commercial tracers, ferucarbotran and Synomag©-D coated with PEG.

The synthesized and commercially obtained SPIONs were investigated using bright field transmission electron microscopy (TEM). Because PEGsilane (RL-1 particles), carboxydextran (ferucarbotran) and PEG-OMe modified dextran (Synomag®-D) coatings give negligible contrast under electron microscopy, the physical morphology and size distribution are representative of the iron oxide crystal cores of each tracer. As shown in FIG. 2.1, the RL-1C particles (A), representative of the RL-1 particles, are single core with narrow size distribution, while ferucarbotran particles (B) consists of small agglomerated cores and Synomag®-D particles (C) consist of heterogeneous nanocrystal clusters. Volume weighted mean physical diameters and standard deviation of all tracers are summarized in Table 1. RL-1 particles had core diameters and standard deviation of 22.6±2.0 nm (RL-1A), 20.7±2.7 nm (RL-1B), and 21.4±2.4 nm (RL-1C). Ferucarbotran had core diameters and standard deviation 9.6±2.9 nm, and Synomag®-D particles had core diameters and standard deviation of 28.6=9.4 am.

TABLE 1
Comparison of physical and magnetic properties and MPI performance
of commercial tracers and RL-1 tracers tailored for MPI.

| | Ferucar- | | | | |
|---|---|---|---|---|---|
| | botran | Synomag ®-D | RL1 | RL2 | RL3 |
| Dₚ, [nm] | 9.6 | 28.6 | 22.6 | 20.7 | 21.4 |
| σₚ, [nm] | 2.9 | 9.4 | 2.0 | 2.7 | 2.4 |
| D_{m, 1}^{a}, [nm] | 7.6 | 8.2 | 2.5 | 3.1 | 2.8 |
| σ_{m, 1}^{a}, [nm] | 4.1 | 3.0 | 1.6 | 1.4 | 1.6 |
| D_{m, 2}^{a}, [nm] | 22.1 | 19.3 | 18.1 | 17.1 | 18.4 |
| σ_{m, 2}^{a}, [nm] | 4.4 | 3.7 | 4.2 | 2.1 | 3.0 |
| ϕ₁ | 0.8 | 0.2 | 0.1 | 0.1 | 0.1 |
| Dₕ, [nm] | 65 | 60 | 54 | 76 | 55 |
| σₕ, [nm] | 28 | 18 | 25 | 35 | 20 |
| ζ, [mV] | −12.9 | −6.5 | — | — | −7.6 |
| Mₛ, [Am²/kg] | 32 | 56 | — | — | 44 |
| T_{B}, [K] | 226 | 320 | — | — | 307 |
| K, [kJ/m³] | 21 | 30 | — | — | 30 |
| RELAX ™ | 25.8 | 87.8 | 77.3 | 69.5 | 82.6 |
| Signal, [μg Fe⁻¹] | | | | | |
| RELAX ™ | 11.2 | 9.2 | 11.4 | 13.0 | 11.9 |
| FWHM, [mT] | | | | | |
| FWHM^{b}, [mm] | 1.96 | 1.61 | 2.00 | 2.28 | 2.09 |

^{a}Magnetic diameter distribution parameters were obtained in DI water suspension.
FWHM [mm] is calculated using the gradient value of 5.7 T/m.

Magnetic properties of the particles are critical for their performance in MPI. Therefore, the magnetic properties of all tracers were evaluated using SQUID magnetometry. As seen in the magnetization versus magnetic field (MH) curves, superparamagnetism is apparent for all tracers either in liquid (FIG. 2.2A, FIG. 2.5.5A, and FIG. 2.5.6A) or in solid matrix (FIG. 2.1B, FIG. 2.5.5B, and FIG. 2.5.6B), as there is a very large initial susceptibility, negligible coercivity or remanent magnetization, and magnetic saturation is reached at relatively small magnetic fields. Superposition of the MH measurements for three different temperatures in solid TEGMA further suggests superparamagnetic behavior (FIG. 2.1B, FIG. 2.5.5B, and FIG. 2.5.6B). The magnetization data was fitted to the Langevin function weighted using a bimodal lognormal size distribution. The results suggest that all three tracers behave as if possessing two populations of particles with different magnetic size distributions. The arithmetic volume-weighted mean magnetic diameter, standard deviation, and volume fraction of each population are summarized in Table 1. Magnetic diameter distributions are also illustrated in FIG. 2.2D (RL-1C) and FIG. 2.5.7 (all tracers studied). The RL-1 particles had consistent magnetic distributions with the majority (ϕ₁˜0.9) of the particles having magnetic diameters of 18.1±4.2 nm, 17.1±2.1 nm, and 18.4±3.0 nm. The remaining ϕ˜0.1 of the particles had a relatively small magnetic diameter of 2.5±1.6 nm, 3.1±1.4 nm, and 2.8±1.6 nm. The larger magnetic diameter population is consistent with the physical size distribution of the RL-1 particles. For ferucarbotran, ϕ₁˜0.81 of the magnetic volume had a size of 7.6±4.1 nm, while the remaining ϕ˜0.19 had a magnetic diameter of 22.1±4.4 nm, consistent with other reports in the literature [33]. The small magnetic diameter fraction in ferucarbotran is consistent with the physical size, although the magnetic diameter distribution is broader. It has been suggested that the population with larger magnetic diameter arises due to magnetic interactions between individual crystals in the dextran matrix [33]. For Synomag®-D, ϕ₁˜0.84 of the magnetic volume had a magnetic diameter of 19.3±3.7 nm and ϕ˜0.16 had a magnetic diameter of 8.2±3.0 nm. These numbers are similar (if slightly smaller) to the physical size of the particle clusters and the small cores that make up each cluster. Interestingly, the larger magnetic size has a narrower distribution relative to the physical size distribution of the clusters that make up Synomag®-D.

The temperature dependence of magnetization was evaluated using zero field cooled/field cooled (ZFC-FC) measurements by measuring the magnetization of a sample embedded in solid matrix as a function of temperature. These measurements yield the blocking temperature of the nanoparticles, indicative of the temperature above which the majority of the particles become superparamagnetic. The resulting ZFC-FC curves are shown in FIG. 2.2C (RL-1C), FIG. 2.5.5C (ferucarbotran), and FIG. 2.5.6C (Synomag®-D). The blocking temperatures were found to be 307 K, 226 K, and 310 K, for RL-1C, ferucarbotran, and Synomag®-D, respectively. The blocking temperatures were then used to calculate the effective anisotropy constant of all tracers. The anisotropy constant contributes to the particle relaxation mechanism and ultimately affects MPI performance [34]. It was found that the effective anisotropy constant of RL-1 (30 kJ/m³) is similar to that of Synomag®-D (30 kJ/m³) and both are larger than ferucarbotran (21 kJ/m³). All three of these values are higher than the Kₘ value for bulk magnetite reported in literature (13.5 kJ/m³) [35]. We note that recent work suggests that true blocking temperatures for particle systems with size and shape polydispersity are significantly lower than the peak of the ZFC curve [36].

The arithmetic volume weighted mean hydrodynamic diameter and standard deviation values of all three nanoparticles were evaluated using dynamic light scattering (DLS) and are summarized in Table 1. RL-1C particles had a hydrodynamic diameter of 55±20 nm, ferucarbotran had a hydrodynamic diameter of 65±28 nm, and Synomag®-D had a hydrodynamic diameter of 60±18 nm. Physical, magnetic, and hydrodynamic size distributions are illustrated in FIG. 2.2D for RL-1C and for all particles in FIG. 2.5.7. The zeta potential was determined at physiological pH of 7.2-7.4. RL-1C particles had a zeta potential of (ξ=−7.6 mV, ferucarbotran had a zeta potential of ξ=−12.9 mV, and Synomag®-D particles had a zeta potential of ξ=−6.5 mV.

Dynamic magnetic susceptibility measurements were made for all tracers to study their relaxation mechanism (FIG. 2.5.8). Magnetic nanoparticles respond to time-varying magnetic fields by internal dipole rotation (i.e., Néel relaxation) or physical particle rotation (i.e., Brownian relaxation), with each mechanism being sensitive to the properties of the particles and the surrounding medium. It is important to characterize the mechanism of SPION magnetic relaxation because this can impact their MPI performance [37]. Because the viscosity of the carrier liquid will affect the relaxation time of particles undergoing Brownian relaxation but not of particles undergoing Néel relaxation, we measured dynamic magnetic susceptibility spectra in water (η=0.001 Pa s) and in water-glycerol solutions (η=0.0152 Pa s) [27]. For particles undergoing Brownian relaxation, one would expect a significant (one-decade) shift toward lower frequency in the DMS spectra of particles in the water-glycerol solution, compared to particles in water. No such shift is observed in the DMS spectra for all tracers, suggesting they all undergo Néel relaxation (FIG. 2.5.8). Furthermore, peaks were not observed at the calculated Brownian peak frequencies (dotted lines in FIG. 2.5.8), further suggesting that the particles undergo primarily Néel relaxation [27].

### Comparison of Tracer MPI Performance

We evaluated the MPI performance of in-house synthesized RL-1 tracers and compared these with commercial ferucarbotran and Synomag®-D. MPI performance was characterized according to the point-spread function (PSF) using the MPI RELAX™ module and by evaluating the limit of detection for a dilution series of samples imaged in 2D high sensitivity multichannel scan mode (FIG. 2.3). The RELAX™ module in the MOMENTUM™ measures the magnetization for a field sweep between −100 mT and 100 mT using a 16 mT, 45 kHz excitation field. The PSF is related to the derivative of the Langevin function and characterizes the performance of SPIONs in x-space MPI [37, 38]. The PSF was used to determine the signal intensity (peak height) per iron mass, which is a measure of expected particle sensitivity in MPI, and to obtain the full-width half-maximum (FWHM), which relates to the expected resolution in MPI. Representative PSF results for all three tracers are shown in FIG. 2.3A. Table 1 summarizes the PSF properties for all particles used in this study. FIG. 2.5.9 shows PSF for three independent batches of RL-1 particles, showing reproducibility in MPI performance. The MPI maximum signal intensity values for the three batches of RL-1 particles range from 70 to 83 μg_{Fe}⁻¹, while the corresponding values are 26 μg_{Fe}⁻¹ for ferucarbotran and 88 μg_{Fe}⁻¹ for Synomag®-D. In terms of resolution, the three batches of RL-1 particles had FWHM ranging from 11.4 mT to 13.0 mT, while the corresponding values were 11.2 mT for ferucarbotran and 9.2 mT for Synomag®-D. The spatial FWHM in units of mm were calculated assuming a field gradient of 5.7 T/m, resulting in expected spatial resolutions of 2.0 to 2.3 mm for RL-1 particles, 2.0 mm for ferucarbotran, and 1.6 mm for Synomag®-D. Using the PSF we observed that RL-1 particles and Synomag®-D have similar sensitivity in the MPI. In terms of resolution, RL-1 and ferucarbotran are similar while Synomag®-D is slightly better.

In contrast to expectations based on the PSF peak intensities, FIG. 2.3B suggests higher signal per unit mass for RL-1C particles imaged in 2D high-sensitivity scan mode (3.055 T/m gradient strength, 15.5 mT RF excitation in the x-channel and 20.5 mT RF excitation in the z-channel). However, although FIG. 2.3B suggests a difference in signal per unit mass, the limit of detection (LoD) calculated using equations (13) and (14) is similar for RL-1C and Synomag®-D nanoparticles. The calculated LoD was 28.31 ng_{Fe} for RL-1C particles, 29.81 ng_{Fe} for Synomag®-D, and 57.75 ng_{Fe} for ferucarbotran. The sensitivity for each tracer was further evaluated by inspection of the z-channel images obtained in the high sensitivity scans for each dilution for each tracer. FIG. 2.5.10 shows these scans. The corresponding mean signal to noise ratio (mSNR) for each tracer and each dilution are shown in FIG. 2.5.11. According to this analysis, the RL-1 sample can still be detected (mSNR=4.5) at 30 ng_{Fe}, while for Synomag®-D at 31 ng_{Fe} we obtained mSNR=1.7 and for Ferucarbotran at 32 ng_{Fe} we obtained mSNR=1.8. For the next larger tracer mass in the series, we obtained mSNR=3.6 for Synomag®-D at 62 ng_{Fe} and mSNR=3.6 for Ferucarbotran at 64 ng_{Fe}. We note that while the two methods of LoD determination agree for RL-1, additional fiducials of intermediate masses would be necessary for Synomag®-D and ferucarbotran.

### Tracer Pharmacokinetics in Mice

The pharmacokinetics of the three tracers was evaluated in mice and blood circulation and liver accumulation half-lives were estimated using single-compartment models. Particles were administered via tail vein injection. In vivo MPI 2D images of each tracer in mice (n=3) were taken at specified time intervals. These images were analyzed by image registration of MPI and CT images and positioning ROIs around the heart and around the liver/spleen (denoted as liver). The MPI total intensity was obtained from the ROIs for each animal for all tracers at all time points. Representative images for one mouse from the RL-1C group for all time points are shown in FIG. 2.5.12.

FIG. 2.4 shows representative MPI scans, registered to CT for anatomical reference, at similar time points and the associated MPI total intensity data for all three animals at all time points for each tracer. Immediately after intravenous injection of the tracer, one should observe whole-body distribution of the tracer, with greater signal intensity in organs with larger blood volume, such as the heart and lungs. Over time, the vascular signal will decrease, while the signal in the liver and spleen will increase. The images of all mice right before injection (t=0 min) show no signal, as expected since there is no tracer. Three minutes after injection (t=3 min), RL-1C tracers were seen to be distributed throughout the body, with the greatest signal corresponding to the heart. Similar distribution was observed for Synomag®-D, albeit with a weaker heart signal and more significant liver signal compared to RL-1C. In contrast, ferucarbotran was observed primarily in the liver and spleen, even at the relatively short time point of t=3 min. One hour after injection, the signal from RL-1C was still primarily in the heart and lungs, whereas almost all the signal for ferucarbotran and Synomag®-D came from the liver and spleen. By 24 hours, signal for all tracers was consistent with accumulation in the liver and spleen. Three-dimensional visualization of the tracers using MPI registered with CT for anatomical reference can be found in Movie S1 for RL-1C at 1 h, Movie S2 for RL-1C at 24 h, Movie S3 for ferucarbotran at 24 h, and Movie S4 for Synomag®-D at 24 h. Three-dimensional MPI images for the two commercial tracers were not acquired at 1 h due to their faster clearance rate relative to the time it takes to obtain a 3D image. Qualitatively, these results suggest that RL-1 particles have longer blood circulation than the commercially available particles.

Blood circulation half-life for all three tracers was calculated from fitting the MPI total signal intensity for the heart and liver ROI to a simple first-order one compartment model. FIG. 2.4 shows the data for each tracer, for all time points collected for each mouse. The shaded region corresponds to the 95% confidence interval for the fit prediction and indicates good fitting for all the tracers. RL-1C showed the longest circulation half-life of t_{1/2,heart}=6.99 h, while ferucarbotran had a t_{1/2,heart}=0.59 h and Synomag®-D had a t_{1/2,heart}=0.62 h. The same algorithm used to calculate blood circulation half-life was used to assess the rate of accumulation of tracers in the liver and spleen. The results were t_{1/2,liver}=7.72 h for RL-1C, t_{1/2,liver}=1.00 h for ferucarbotran, and t_{1/2,liver}=0.58 h for Synomag®-D. Both qualitatively and quantitatively, RL-1 tracers had a much longer blood circulation half-life than the commercial tracers ferucarbotran and Synomag®-D coated with PEG.

### DISCUSSION

The results of this study show how thermal decomposition of iron-oleate precursor in the presence of molecular oxygen can be leveraged to obtain single-core SPIONs with reproducible enhanced MPI performance. The three tracers studied had similar hydrodynamic diameters according to dynamic light scattering, but consisted of SPIONs with different crystal sizes and morphology. Interestingly, although RL-1 consisted of ˜20-22 nm single-core crystals and Synomag®-D consisted of ˜29 nm multicore crystals, they had similar magnetic diameter distributions, blocking temperatures, and estimated effective anisotropy constants. We believe this is the reason why they had similar MPI sensitivity performance, albeit with better expected resolution for Synomag®-D. This is consistent with the notion that MPI performance is determined principally by the magnetic properties of the tracer. Of relevance, both RL-1 and Synomag®-D had the majority (80-90%) of their magnetic volume assigned to a population with a magnetic diameter of ˜18-19 nm, with the rest of the magnetic volume corresponding to a population with a magnetic diameter of ˜2-4 nm. In contrast, only 20% of the magnetic volume in ferucarbotran corresponded to a population with a magnetic diameter of ˜22 nm, with the rest of the magnetic volume corresponding to a population with a magnetic diameter of ˜4.4 nm. We believe this is the reason why RL-1 and Synomag®-D have ˜3×better MPI signal per Fe mass than ferucarbotran.

Ideal tracers for blood pool imaging using MPI should yield sufficient vascular signal after a single tracer injection for a period long enough to allow diagnosis. Long circulating nanoparticles can lead to better planning and diagnostics for different applications such as cancer detection and imaging, blood pool evaluation, monitoring bleeding, and for functional MPI in the brain. Blood circulation half-life was determined for RL-1 and the two commercially available tracers (ferucarbotran and Synomag®-D) in female Balb/c mice. The pharmacokinetic data was fitted to a standard first-order one compartment model. The assumptions of the one compartment model are that the tracer distributes and equilibrates rapidly throughout the vascular system and that elimination begins immediately after administration. The results shown in FIG. 2.4 appear consistent with a one compartment model. Importantly, RL-1C showed the longest blood circulation half-life of ˜7 hours.

Ferucarbotran has been studied widely as a contrast agent for MRI. In another MPI study performed in rats using ferucarbotran, no signal was observed in the heart or jugular veins 10 minutes after injection, suggesting that the blood circulation half-life of ferucarbotran in rats is much shorter than 10 minutes [20]. The blood circulation half-life of ferucarbotran has also been evaluated using magnetic particle spectroscopy of blood samples in mice and rabbits, and the half-life was determined to be 5-10 minutes [39, 40]. However, that study was limited due to the small amount of blood that could be obtained and the sensitivity of the equipment used to detect tracer signal. In a human study of ferucarbotran as an MRI contrast agent, the tracer exhibited a biexponential blood concentration decay, with a half-life of 3.9-5.8 min for the fast initial phase accounting for roughly 80% of the injected dose and a second half-life of 2.4-3.6 h for the second phase [41]. In another study using the Bruker pre-clinical MPI scanner, which is able to perform scans at 21.5 milliseconds per frame, the authors observed biexponential blood concentration decay for ferucarbotran in FVB mice with first fast clearance phase of 0.63 minute and a slower phase of 13 minutes [42]. We did not observe biexponential decay in blood tracer concentration. However, by our first imaging time point, 3 minutes after injection, most of the ferucarbotran seemed to have accumulated in the liver already. As such, our measurements most likely missed an initial, fast signal decay. This would suggest that our analysis of the data obtained is representative of the second phase of ferucarbotran clearance from blood, which would be in agreement with the study by Kaul et al.[42], which suggests that the second decay phase starts within 3 minutes. Furthermore, it is relevant to point out that the studies by Hamm et al. [41] were in humans, whereas that of Kaul et al. and our study were in mice. It has been shown that nanoparticle tracers have longer blood circulation times in humans than in mice, as it takes about one minute for the tracer to pass the whole circulatory system in a human, while in mice it takes about 5-10 seconds [43].

Use of Synomag®-D in MPI is growing, as they provide better MPI performance than ferucarbotran in terms of sensitivity and resolution. However, the blood circulation time of Synomag®-D particles has not been reported. Our results suggest that Synomag®-D has a blood circulation half-life of 37 minutes, compared to 31 minutes for ferucarbotran.

Dynamic light scattering and zeta potential measurements suggests that all three tracers have similar hydrodynamic size distributions (˜65 nm for ferucarbotran, ˜60 nm for Synomag®-D, and ˜55-75 nm for RL-1) and negative charge (−12.9 mV for ferucarbotran, −6.5 mV for Synomag®-D, and −7.6 mV for RL-1). However, the three tracers differ significantly in terms of the nature of their surface coating. Depending on their coating, nanoparticles can be recognized by the mononuclear phagocytic system and taken up and removed from circulation. Ferucarbotran is coated with carboxymethyl dextran, which is a complex carbohydrate that can easily be phagocytosed by macrophages due to its overall negative charge and due to the action of mannose/lectin receptors, which recognizes the nanoparticle and initiate endocytosis. Coating nanoparticles with PEG is widely adopted to prolong their blood circulation time. Although Synomag®-D is advertised as coated with PEG (25 kDa) for prolonged circulation time in blood, our results suggest a relatively short blood circulation half-life of ˜0.62 hours. We attribute this to the fact that, based on information provided by the supplier, Synomag®-D is actually initially coated with a dextran shell before conjugating PEG onto the dextran. Depending on the extent of coating, the dextran layer on Synomag®-D may be recognized by macrophages and taken up just like in ferucarbotran. In contrast, the RL-1 nanoparticles are coated with a covalently bonded brush of PEG, which we believe is responsible for their comparatively longer blood circulation time.

A possible limitation of the present study is the reliance on MPI signal in regions of interest in vivo to estimate SPION blood circulation and liver/spleen accumulation dynamics, without comparison to ex vivo quantification by other means, such as inductively coupled plasma mass spectroscopy. However, correlation between in vivo and ex vivo MPI SPION quantification and linearity of MPI signal with SPION concentration have been demonstrated previously [12, 20]. While comparison to other means of quantification of iron, such as inductively coupled plasma mass spectrometry, would be desirable, this would require animal euthanasia at each time point of interest, significantly increasing the burden of research animal use. In fact, this is precisely one of the reasons why MPI is so attractive for quantitative tracking of SPIONs in vivo.

### CONCLUSIONS

Magnetic particle imaging, an emerging molecular imaging modality, has great potential in applications such as blood pool imaging, functional brain imaging, cancer imaging, evaluating traumatic brain injury, and in vivo gut bleed detection. Long circulating nanoparticles can lead to better planning and diagnostics for these applications. In this study, MPI-tailored SPION tracers were synthesized through thermal decomposition with molecular oxygen, followed by coating with covalently bonded PEG. Physical and magnetic properties of the synthesized tracers were evaluated and compared to commercial tracers (ferucarbotran and Synomag®-D). The synthesized tracer RL-1 had similar MPI performance compared to Synomag®-D, which was attributed to their similar magnetic diameter distributions and blocking temperatures. Both tracers were ˜3-times better in MPI signal per tracer mass than ferucarbotran result. The blood circulation half-life of the RL-1 tracer was also evaluated and compared to the two commercially available tracers. Analysis of in-vivo MPI in mice study suggests that RL-1 has a blood circulation half-life of 6.99 h, much longer than that of ferucarbotran (0.59 h) and Synomag®-D (0.62 h). These results suggest that RL-1 tracers are excellent candidates for MPI applications that require long blood circulation.

### Abbreviations

SPION: superparamagnetic iron oxide nanoparticle; PEG: polyethyelene glycol; MPI: magnetic particle imaging; AMF: alternating magnetic field; FFR: field free region; FOV: field of view; MRI: magnetic resonance imaging; MPS: magnetic particle spectrometer; APS: 3-aminopropyl triethoxysilane; m-PEG: polyethylene glycol monomethyl ether; TEGDMA: tetra(ethylene glycol) dimethacrylate; EDC: 1-ethyl-3-(3-dimethylaminopropyl) carbodiimide; sulfo-NHS: N-hydroxysulfosuccinimide; SS: stainless steel; PEGsilane: polyethylene glycol-silane conjugate; mPEG-COOH: mPEG acetic acid; NMR: nuclear magnetic resonance; GPC: gel permeation chromatography; MH: magnetization versus magnetic field; ZFC-FC: zero field cooled and field cooled; 3D: three-dimensional; LoD: limit of detection; ROI: region of interest; LoB: limit of blank; PSF: point spread function; TEM: transmission electron microscopy; DLS: dynamic light scattering; FWHM: full-width half-maximum; mSNR: mean signal to noise ratio

### REFERENCES FOR EXAMPLE 2

1. Gleich B, Weizenecker R. Tomographic imaging using the nonlinear response of magnetic particles. Nature. 2005; 435: 1214-7.
2. Goodwill P W, Saritas E U, Croft L R, Kim T N, Krishnan K M, Schaffer D V, et al. X-Space MPI: Magnetic Nanoparticles for Safe Medical Imaging. Advanced Materials. 2012; 24: 3870-7.
3. Zheng B, von See M P, Yu E, Gunel B, Lu K, Vazin T, et al. Quantitative Magnetic Particle Imaging Monitors the Transplantation, Biodistribution, and Clearance of Stem Cells In Vivo. Theranostics. 2016; 6: 291-301.
4. Fuller E G, Sun H, Dhavalikar R D, Unni M, Scheutz G M, Sumerlin B S, et al. Externally Triggered Heat and Drug Release from Magnetically Controlled Nanocarriers. Acs Applied Polymer Materials. 2019; 1: 211-20.
5. Tay Z W, Chandrasekharan P, Chiu-Lam A, Hensley D W, Dhavalikar R, Zhou X Y, et al. Magnetic Particle Imaging-Guided Heating in Vivo Using Gradient Fields for Arbitrary Localization of Magnetic Hyperthermia Therapy. Acs Nano. 2018; 12: 3699-713.
6. Gil C J, Tomov M L, Theus A S, Cetnar A, Mahmoudi M, Serpooshan V. In Vivo Tracking of Tissue Engineered Constructs. Micromachines. 2019; 10.
7. Orendorff R, Peck A J, Zheng B, Shirazi S N, Ferguson R M, Khandhar A P, et al. First in vivo traumatic brain injury imaging via magnetic particle imaging. Physics in Medicine and Biology. 2017; 62: 3501-9.
8. Yu E Y, Chandrasekharan P, Berzon R, Tay Z W, Zhou X Y Y, Khandhar A P, et al. Magnetic Particle Imaging for Highly Sensitive, Quantitative, and Safe in Vivo Gut Bleed Detection in a Murine Model. Acs Nano. 2017; 11: 12067-76.
9. Wu L C, Zhang Y, Steinberg G, Qu H, Huang S, Cheng M, et al. A Review of Magnetic Particle Imaging and Perspectives on Neuroimaging. American Journal of Neuroradiology. 2019; 40: 206-12.
10. Chandrasekharan P, Tay Z W, Zhou X Y, Yu E, Orendorff R, Hensley D, et al. A perspective on a rapid and radiation-free tracer imaging modality, magnetic particle imaging, with promise for clinical translation. British Journal of Radiology. 2018; 91.
11. Herb K, Mason E, Mattingly E, Mandeville J, Mandeville E, Cooley C, et al. Functional MPI (fMPI) of hypercapnia in rodent brain with MPI time-series imaging. International Journal on Magnetic Particle Imaging. 2020; 6.
12. Yu E Y, Bishop M I, Zheng B, Ferguson R M, Khandhar A P, Kemp S J, et al. Magnetic Particle Imaging: A Novel in Vivo Imaging Platform for Cancer Detection. Nano Lett. 2017; 17: 1648-54.
13. Herz S, Vogel P, Kampf T, Dietrich P, Veldhoen S, Ruckert M A, et al. Magnetic Particle Imaging-Guided Stenting. J Endovascular Ther. 2019; 26: 512-9.
14. Nejadnik H, Pandit P, Lenkov O, Lahiji A P, Yerneni K, Daldrup-Link H E. Ferumoxytol Can Be Used for Quantitative Magnetic Particle Imaging of Transplanted Stem Cells. Molecular Imaging and Biology. 2019; 21: 465-72.
15. Eberbeck D, Dennis C L, Huls N F, Krycka K L, Gruttner C, Westphal F. Multicore Magnetic Nanoparticles for Magnetic Particle Imaging. Ieee Transactions on Magnetics. 2013; 49: 269-74.
16. Griese F, Knopp T, Gruettner C, Thieben F, Muller K, Loges S, et al. Simultaneous Magnetic Particle Imaging and Navigation of large superparamagnetic nanoparticles in bifurcation flow experiments. Journal of Magnetism and Magnetic Materials. 2020; 498.
17. Antonelli A, Szwargulski P, Scarpa E S, Thieben F, Cordula G, Ambrosi G, et al. Development of long circulating magnetic particle imaging tracers: use of novel magnetic nanoparticles and entrapment into human erythrocytes. Nanomedicine. 2020; 15: 739-53.
18. Paysen H, Loewa N, Stach A, Wells J, Kosch O, Twamley S, et al. Cellular uptake of magnetic nanoparticles imaged and quantified by magnetic particle imaging. Scientific Reports. 2020; 10.
19. Khandhar A P, Keselman P, Kemp S J, Ferguson R M, Goodwill P W, Conolly S M, et al. Evaluation of PEG-coated iron oxide nanoparticles as blood pool tracers for preclinical magnetic particle imaging. Nanoscale. 2017; 9: 1299-306.
20. Keselman P, Yu E Y, Zhou X Y Y, Goodwill P W, Chandrasekharan P, Ferguson R M, et al. Tracking short-term biodistribution and long-term clearance of SPIO tracers in magnetic particle imaging. Physics in Medicine and Biology. 2017; 62: 3440-53.
21. Unni M, Uhl A M, Savliwala S, Savitzky B H, Dhavalikar R, Garraud N, et al. Thermal Decomposition Synthesis of Iron Oxide Nanoparticles with Diminished Magnetic Dead Layer by Controlled Addition of Oxygen. Acs Nano. 2017; 11: 2284-303.
22. Barrera C, Herrera A P, Bezares N, Fachini E, Olayo-Valles R, Hinestroza J P, et al. Effect of poly(ethylene oxide)-silane graft molecular weight on the colloidal properties of iron oxide nanoparticles for biomedical applications. Journal of Colloid and Interface Science. 2012; 377: 40-50.
23. Zhu M Q, Chang E, Sun J T, Drezek R A. Surface modification and functionalization of semiconductor quantum dots through reactive coating of silanes in toluene. Journal of Materials Chemistry. 2007; 17: 800-5.
24. Chiu-Lam A, Staples E, Pepine C J, Rinaldi C. Perfusion, cryopreservation, and nanowarming of whole hearts using colloidally stable magnetic cryopreservation agent solutions. Science Advances. 2021; 7.
25. Schindelin J, Arganda-Carreras I, Frise E, Kaynig V, Longair M, Pietzsch T, et al. Fiji: an open-source platform for biological-image analysis. Nature Methods. 2012; 9: 676-82.
26. International A. Standard test method for iron in trace quantities using the 1,10-phenanthroline method E394-15. West Conshohocken, PA; 2015.
27. Maldonado-Camargo L, Unni M, Rinaldi C. Magnetic Characterization of Iron Oxide Nanoparticles for Biomedical Applications. Methods in molecular biology (Clifton, NJ). 2017; 1570: 47-71.
28. Chantrell R W, Popplewell J, Charles S W. MEASUREMENTS OF PARTICLE-SIZE DISTRIBUTION PARAMETERS IN FERROFLUIDS. Ieee Transactions on Magnetics. 1978; 14: 975-7.
29. Birks J B. THE PROPERTIES OF FERROMAGNETIC COMPOUNDS AT CENTIMETRE WAVELENGTHS. Proceedings of the Physical Society of London Section B. 1950; 63: 65-74.
30. Fedorov A, Beichel R, Kalpathy-Cramer J, Finet J, Fillion-Robin J C, Pujol S, et al. 3D Slicer as an image computing platform for the Quantitative Imaging Network. Magnetic Resonance Imaging. 2012; 30: 1323-41.
31. Slicer 4.10.2 released.; 2019.
32. Armbruster D A, Pry T. Limit of blank, limit of detection and limit of quantitation. Clin Biochem Rev. 2008; 29 Suppl 1: S49-S52.
33. Eberbeck D, Wiekhorst F, Wagner S, Trahms L. How the size distribution of magnetic nanoparticles determines their magnetic particle imaging performance. Applied Physics Letters. 2011; 98.
34. Zhao Z Y, Garraud N, Arnold D P, Rinaldi C. Effects of particle diameter and magnetocrystalline anisotropy on magnetic relaxation and magnetic particle imaging performance of magnetic nanoparticles. Physics in Medicine and Biology. 2020; 65.
35. Goya G F, Berquó, Fonseca F C, Morales M P. Static and dynamic magnetic properties of spherical magnetite nanoparticles. Journal of Applied Physics. 2003; 94: 3520-8.
36. Livesey K L, Ruta S, Anderson N R, Baldomir D, Chantrell R W, Serantes D. Beyond the blocking model to fit nanoparticle ZFC/FC magnetisation curves. Scientific Reports. 2018; 8: 11166.
37. Croft L R, Goodwill P W, Conolly S M. Relaxation in x-space magnetic particle imaging. IEEE Trans Med Imaging. 2012; 31: 2335-42.
38. Dhavalikar R, Hensley D, Maldonado-Camargo L, Croft L R, Ceron S, Goodwill P W, et al. Finite magnetic relaxation in x-space magnetic particle imaging: Comparison of measurements and ferrohydrodynamic models. J Phys D Appl Phys. 2016; 49.
39. Khandhar A P, Ferguson R M, Arami H, Krishnan K M. Monodisperse magnetite nanoparticle tracers for in vivo magnetic particle imaging. Biomaterials. 2013; 34: 3837-45.
40. Haegele J, Duschka R L, Graeser M, Schaecke C, Panagiotopoulos N, Ludtke-Buzug K, et al. Magnetic particle imaging: kinetics of the intravascular signal in vivo. International Journal of Nanomedicine. 2014; 9: 4203-9.
41. Hamm B, Staks T, Tapuitz M, Maibauer R, Speidel A, Huppertz A, et al. CONTRAST-ENHANCED MR-IMAGING OF LIVER AND SPLEEN—1ST EXPERIENCE IN HUMANS WITH A NEW SUPERPARAMAGNETIC IRON-OXIDE. Jmri-Journal of Magnetic Resonance Imaging. 1994; 4: 659-68.
42. Kaul M G, Mummert T, Jung C, Salamon J, Khandhar A P, Ferguson R M, et al. In vitro and in vivo comparison of a tailored magnetic particle imaging blood pool tracer with Resovist. Physics in Medicine and Biology. 2017; 62: 3454-69.
43. Hall C, Lueshen E, Mosat A, Linninger A A. Interspecies scaling in pharmacokinetics: A novel whole-body physiologically based modeling framework to discover drug biodistribution mechanisms in vivo. Journal of Pharmaceutical Sciences. 2012; 101: 1221-41.

It should be noted that ratios, concentrations, amounts, dimensions, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a range of “about 0.1% to about 5%” should be interpreted to include not only the explicitly recited range of about 0.1% to about 5%, but also include individual ranges (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to the numerical value and measurement technique. In addition, the phrase “about ‘x’ to ‘y’ includes “about ‘x’ to about ‘y′″.

It should be emphasized that the above-described embodiments of this disclosure are merely possible examples of implementations, and are set forth for a clear understanding of the principles of this disclosure. Many variations and modifications may be made to the above-described embodiments of this disclosure without departing substantially from the spirit and principles of this disclosure. All such modifications and variations are intended to be included herein within the scope of this disclosure and protected by the following claims.

1. A composition, comprising a coated magnetic nanoparticle having:
a magnetic core;
a first poly(ethylene glycol) (PEG)-silanization coating covalently attached to the magnetic core, wherein the first PEG-silanization coating comprises a mixture of PEG silane and aminosilane; and
a second PEG coating covering at least a part of the first PEG-silanization coating, wherein the second PEG coating comprises a PEG group having at least one amine reactive group, the second PEG coating is attached to the first PEG-silanization coating via an amino group on the aminosilane and the amine reactive group on the second PEG coating.

2. The composition of claim 1, wherein no primary amines can be detected on the surface of the nanoparticle using a standard assay.

3. The composition of claim 1, wherein molar ratio of PEG silane and aminosilane is about 2:1 to 1:2.

4. The composition of claim 1, wherein the amine reactive group is selected from the group consisting of: a carboxy group, and a succinimidyl ester group.

5. The composition of claim 1, wherein the PEG group of the second PEG coating includes a secondary PEG moiety attached to the end opposite of the PEG group as the amine reactive group, wherein the secondary PEG moiety is selected from a thiol reactive group, a click reactive group, a fluorophore, a peptide, a targeting agent, and a combination of any of these.

6. The composition of claim 1, wherein the magnetic core comprises at least one selected from the group consisting of iron oxide, magnetite, and substituted ferrite, wherein the substituted ferrite is selected from the group consisting of nickel ferrite, aluminum ferrite, manganese ferrite, zinc ferrite, cobalt ferrite, and combinations thereof.

7. The composition of claim 6, wherein the magnetic core is superparamagnetic.

8. The composition of claim 1, wherein the coated magnetic nanoparticle has a diameter of from about 3 nm to about 100 nm.

9. The composition of claim 1, wherein the aminosilane is selected from the group consisting of 3-aminopropyltrimethoxysilane, 3-aminopropylmethyldimethoxysilane, 3-aminopropyldimethylmethoxysilane, N-(2-aminoethyl)-3-aminopropylmethyldimethoxysilane, N-(2-aminoethyl-3-aminopropyl)trimethoxysilane, 4-aminobutyldimethylmethoxysilane, 4-aminobutyltrimethoxysilane, aminoethylaminomethylphenethyltrimethoxysilane, N-(2-aminoethyl)-3-aminoisobutylmethyldimethoxysilane, N-(6-aminohexyl)aminopropyltrimethoxysilane, 3-(m-aminophenoxy)propyltrimethoxysilane, aminophenyltrimethoxysilane, 3-aminopropyltriethoxysilane, 3-aminopropylmethyldiethoxysilane, 3-aminopropyldimethylethoxysilane, N-(2-aminoethyl)-3-aminopropylmethyldiethoxysilane, N-(2-aminoethyl-3-aminopropyl)triethoxysilane, 4-aminobutyldimethylethoxysilane, 4-aminobutyltriethoxysilane, aminoethylaminomethylphenethyl triethoxysilane, N-(2-aminoethyl)-3-aminoisobutylmethyldiethoxysilane, N-(6-aminohexyl)aminopropyltriethoxysilane, 3-(m-aminophenoxy)propyltriethoxysilane, aminophenyltriethoxysilane, and combinations thereof.

10. The composition of claim 1, wherein first PEG-silanization coating comprises the formula: 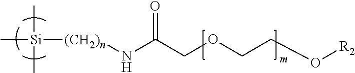
wherein the Si is bonded directly or indirectly to the magnetic core;
wherein R2 is selected from H or a C1-C6 alkyl group;
n is 1, 2, 3, 4, 5, or 6;
m is an integer from about 10 to about 1000.

11. The composition of claim 10, wherein R2 is methyl, and n is 3.

12. The composition of claim 1, further comprising a cryopreservation agent is selected from the group consisting of VS55, DP6, and glycerol.

13. A method for imaging a subject comprising:
introducing the composition of claim 1 to the subject; and
imaging the subject with an imaging technique.

14. The method of claim 13, further comprising applying an alternating magnetic field to the subject.

15-19. (canceled)

20. A method for producing a coated magnetic nanoparticle comprising:
subjecting a magnetic core to a first composition comprising a mixture of poly(ethylene glycol)-silane (PEG-silane) group and aminosilane to form a first nanoparticle coated with a first PEG-silanization coating comprising a first ligand derived from the PEG-silane group, wherein the first ligand is covalently attached to the magnetic core, wherein the aminosilane is covalently bonded to the magnetic core; and
subjecting the first nanoparticle to a second composition comprising a PEG group to form a coated magnetic nanoparticle further coated with a second layer comprising PEG group, wherein the PEG group having at least one amine reactive group, wherein the second PEG coating is attached to the first PEG-silanization coating via an amino group on the aminosilane and the amine reactive group on the second PEG coating.

21. The method of claim 20, wherein no primary amines can be detected on a surface of the second nanoparticle using a standard assay.

22. The method of claim 20, wherein the magnetic core comprises at least one selected from the group consisting of iron oxide, magnetite, and substituted ferrite, wherein the substituted ferrite is selected from the group consisting of nickel ferrite, aluminum ferrite, manganese ferrite, zinc ferrite, cobalt ferrite, and combinations thereof.

23. The method of claim 20, wherein the aminosilane is selected from the group consisting of 3-aminopropyltrimethoxysilane, 3-aminopropylmethyldimethoxysilane, 3-aminopropyldimethylmethoxysilane, N-(2-aminoethyl)-3-aminopropylmethyldimethoxysilane, N-(2-aminoethyl-3-aminopropyl)trimethoxysilane, 4-aminobutyldimethylmethoxysilane, 4-aminobutyltrimethoxysilane, aminoethylaminomethylphenethyltrimethoxysilane, N-(2-aminoethyl)-3-aminoisobutylmethyldimethoxysilane, N-(6-aminohexyl)aminopropyltrimethoxysilane, 3-(m-aminophenoxy)propyltrimethoxysilane, aminophenyltrimethoxysilane, 3-aminopropyltriethoxysilane, 3-aminopropylmethyldiethoxysilane, 3-aminopropyldimethylethoxysilane, N-(2-aminoethyl)-3-aminopropylmethyldiethoxysilane, N-(2-aminoethyl-3-aminopropyl)triethoxysilane, 4-aminobutyldimethylethoxysilane, 4-aminobutyltriethoxysilane, aminoethylaminomethylphenethyl triethoxysilane, N-(2-aminoethyl)-3-aminoisobutylmethyldiethoxysilane, N-(6-aminohexyl)aminopropyltriethoxysilane, 3-(m-aminophenoxy)propyltriethoxysilane, aminophenyltriethoxysilane, and combinations thereof.

24. The method of claim 20, wherein the PEG-silane has an alkoxy group on a terminus of the PEG moiety opposite a terminus conjugated to a silane moiety.

25. The method of claim 24, wherein the PEG-silane has the formula: 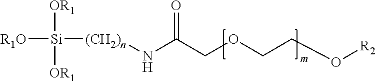
wherein R1 is H or a C1-C6 alkyl group;
R2 is H or a C1-C6 alkyl group;
n is 1, 2, 3, 4, 5, or 6; and
m is an integer from about 10 to about 1000.

26-28. (canceled)
